(19) United States
(12) Patent Application Publication
(10) Pub. No.: US 2021/0355086 A1
(43) Pub. Date: Nov. 18, 2021

(54) PROCESS FOR PREPARING A NOVEL PHENICOL ANTIBACTERIAL AGENT

(71) Applicant: Zoetis Services LLC, Parsippany, NJ (US)

(72) Inventors: Randall Lee DeJong, Kalamazoo, MI (US); Gregory Lee Karrick, Portage, MI (US); Timothy Lee Stuk, Mattawan, MI (US)

(21) Appl. No.: 17/278,385

(22) PCT Filed: Sep. 23, 2019
(86) PCT No.: PCT/US2019/052336

§ 371 Date: Mar. 22, 2021

Related U.S. Application Data
(60) Provisional application No. 62/735,353, filed on Sep. 24, 2018.

(51) Int. Cl. C07D 213/71; B01J 31/04; B01J 31/24

(52) U.S. Cl. CPC C07D 213/71; B01J 31/04; B01J 2531/824; C07B 2200/07; B01J 31/2452

(57) ABSTRACT

The present invention provides a novel process for preparing a diastereomeric mixture of the phenicol of Formula (1), its use for the treatment of bacterial infections in animals, and veterinary compositions containing the Formula (1) compound, diastereomers thereof, and veterinary acceptable salts thereof.

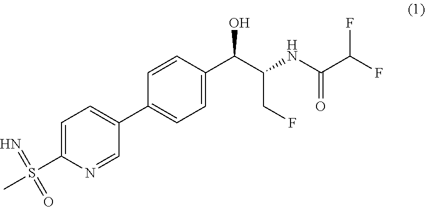 [SMILES: CS(=N)(=O)c1ccc(-c2ccc([C@@H](O)[C@@H](CF)NC(=O)C(F)F)cc2)cn1]

### FIELD OF THE INVENTION

The present invention provides a process for preparing a novel phenicol of Formula (1), and diastereomers thereof

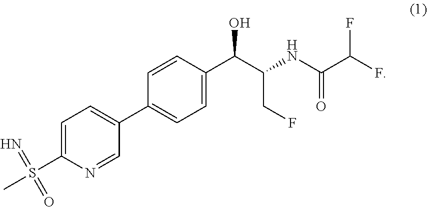 [SMILES: CS(=N)(=O)c1ccc(-c2ccc([C@@H](O)[C@@H](CF)NC(=O)C(F)F)cc2)cn1]

### BACKGROUND OF THE INVENTION

Florfenicol, also referred to as 3-fluorothiamphenicol, is a veterinary broad-spectrum chloramphenicol antibiotic having biological activities against a variety of Gram-positive bacteria and Gram-negative bacteria. Compared with thiamphenicol, florfenicol has higher antibacterial activity, broader antibacterial spectrum, better absorption and less adverse reactions, and the antibacterial activity of florfenicol is up to 10 times that of thiamphenicol. Florfenicol can be used in the treatment of bovine, porcine, avian and other animals with bacterial and mycotic diseases, as well as in the preparation of aquaculture drugs.

In recent years, many genera and species of bacteria began to show certain resistance to florfenicol. For example, Salmonella (Bolton, L. F. et al., Clin. Microbiol., 1999, 37, 1348); E. coli (Keyes, K. et al., Antimicrob. Agents Chemother., 2000, 44, 421); Klebsiella pneumoniae (Cloeckaert, A. et al., Antimicrob. Agents Chemother., 2001, 45, 2381); and a water-borne pathogen Photobacterium damselae subsp. piscicida (Kim, E. et al., Microbiol. Immunol., 1996, 40, 665) were found to be resistant to florfenicol. The emergence of the resistance to florfenicol along with the risk of its spread have contributed to the need for new antibiotics that can retain or exceed the activity of florfenicol. WO2014172443A1 disclosed a new phenicol antibiotic of Formula (1) having the structure below:

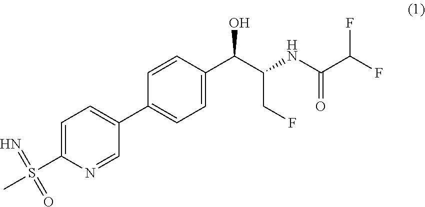 [SMILES: CS(=N)(=O)c1ccc(-c2ccc([C@@H](O)[C@@H](CF)NC(=O)C(F)F)cc2)cn1]

which is 2,2-difluoro-N-((1R,2S)-3-fluoro-1-hydroxy-1-(4-(6-(S-methylsulfonimidoyl)pyridin-3-yl)phenyl)propan-2-yl)acetamide. This application describes an alternative method for preparing the compound of Formula (1), its diastereomers, and the Formula (A) compound. A key intermediate in the preparation of the phenicol of Formula (1) is the Formula (A) compound, (5-bromopyridin-2-yl)(imino)(methyl)-A6-sulfanone, hydrochloride.

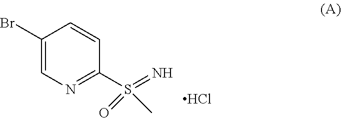

The free base of the Formula (A) compound was previously described in WO2014/172443. A second key intermediate to preparing the compound of Formula (1) is the Formula (B) compound, N-((1R,2S)-1-(4-chlorophenyl)-3-fluoro-1-hydroxypropan-2-yl)-2,2-difluoroacetamide. Preparation of the chiral Formula (B) compound was described in CN106631872A.

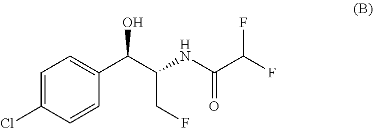 [SMILES: O=C(N[C@H](CF)[C@H](O)c1ccc(Cl)cc1)C(F)F]

This application provides a new chemical synthetic route for the preparation of about a 1:1 diastereomeric mixture of Formula (1) by coupling the key intermediates (racemic Formula (A) and enantiomerically pure Formula (B)), which has fewer chemical steps and higher yields than previously described processes. This application also provides a new chemical synthesis route for the preparation of Formula (A) which also has fewer chemical steps and higher yields.

### SUMMARY OF THE INVENTION

In view of the number of procedural steps for preparing the compound of Formula (1) with low yields, a more robust and efficient process was required, particularly for preparing about a 1:1 diastereomeric mixture of the Formula (1) compound. The 1:1 diastereomeric mixture of Formula (1) is the preferred combination (Formula's 1a:1b) for production manufacturing, regulatory control, and consistent drug formulation for antibacterial clinical efficacy. As described herein, the Formula (1) compound is a diastereomeric mixture of 2,2-difluoro-N-((1R,2S)-3-fluoro-1-hydroxy-1-(4-(6-((S)—S-methylsulfonimidoyl)pyridin-3-yl)phenyl)propan-2-yl)acetamide (Formula (1a)) and 2,2-difluoro-N-((1R,2S)-3-fluoro-1-hydroxy-1-(4-(6-((R)—S-methylsulfonimidoyl)pyridin-3-yl)phenyl)propan-2-yl)acetamide (Formula (1b)), shown below.

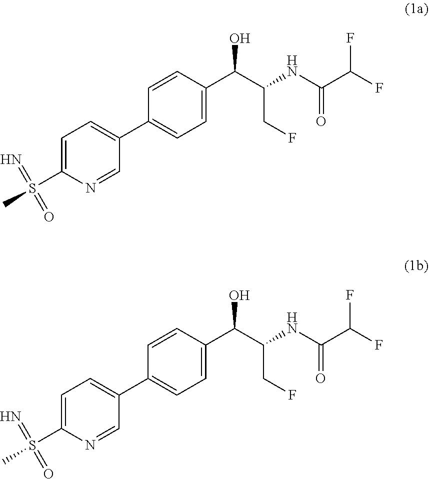 [SMILES: C[S@](=N)(=O)c1ccc(-c2ccc([C@@H](O)[C@@H](CF)NC(=O)C(F)F)cc2)cn1 ; C[S@@](=N)(=O)c1ccc(-c2ccc([C@@H](O)[C@@H](CF)NC(=O)C(F)F)cc2)cn1]

Preferrably, the diastereomers of Formula (1a) and Formula (1b) are prepared in a diastereomeric mixture in a ratio of about 48:52 (1a:1b) to 52:48 (1a:1b); and preferably from about 49:51 (1a:1b) to 51:49 (1a:1b). The following procedural steps provide the synthetic steps for preparing the diastereomeric mixture of Formula (1).

In one aspect of the invention, is a process for preparing the crude compound of Formula (1) by coupling the racemic Formula (A) with the enantiomerically pure Formula (B). In another aspect of the invention, is a process for preparing the crude compound of Formula (1) by coupling the racemic Formula (A) with the enantiomerically pure Formula (B); and then purifying the crude Formula (1) compound to prepare the 1:1 diastereomeric mixture of Formula (1). In another aspect of the invention is a process for preparing the enantiomeric pure Formula (B). In yet another aspect of the invention is a process of preparing the racemic Formula (A).

In one aspect of the invention, is a process for preparing the Formula (1) compound (crude)

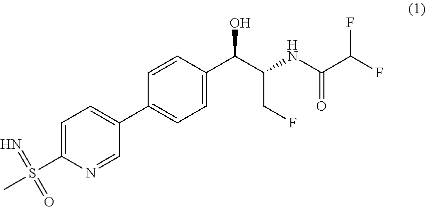 [SMILES: CS(=N)(=O)c1ccc(-c2ccc([C@@H](O)[C@@H](CF)NC(=O)C(F)F)cc2)cn1]

comprising the steps of:
a) pre-activating the Formula (B) compound with a palladium catalyst in the presence of an alcohol, a ligand, and a borylation buffer;

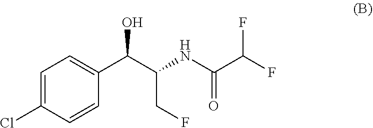 [SMILES: O=C(N[C@H](CF)[C@H](O)c1ccc(Cl)cc1)C(F)F]

b) borylating the preactivated Formula (B) compound of Step (a) by adding a borylating agent in an alcohol to the reactants of Step (a);
c) mixing the Formula (A) compound with a base in an alcohol, co-solvent, or mixtures thereof; and

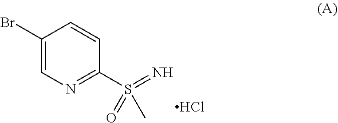

d) mixing the reactants of Step (c) to the reactants of Step (b) to give the compound of Formula (1), and diastereomers thereof.

In another aspect of the invention, is a process for preparing the Formula (1) compound (crude), comprising the steps of:
a) pre-activating the Formula (B) compound with a palladium catalyst selected from the group consisting of Pd(OAc)₂, PdCl₂, Pd-G2-XPhos, Pd-XPhos Crotyl Cl, Pd(dppf)Cl₂, Pd-G2-PCy₃, and Pd₂(dba)₃, in the presence of an alcohol, a ligand selected from the group consisting of XPhos, SPhos, dppp, dppf, dba, PPh₃, and PCy₃, and a borylation buffer that comprises an acid and a base, wherein said acid is selected from the group consisting of HOAc, citric acid, formic acid, chloroacetic acid, and ammonium acetate;
b) borylating the preactivated Formula (B) compound of step (a) by adding a borylating agent in an alcohol wherein the borylating agent is selected from the group consisting of bisboronic acid, bisboronic acid and ethylene glycol, bisboronic acid and propylene glycol, B₂Pin₂, and B₂(NMe₂)₄;
c) mixing the Formula (A) compound with a base in an alcohol, co-solvent, or mixtures thereof; and
d) mixing the reactants of Step (c) to the reactants of Step (b) to give the compound of Formula (1), and diastereomers thereof.

In yet another aspect of the invention, the base in Step (a) and (c) is selected from the group consisting of KOAc, CsOAc, TEA, K₂CO₃, Na₂CO₃, Cs₂CO₃, DIPEA, and K₃PO₄, and mixtures thereof. In yet another aspect of the invention, the base in Step (a) and (c) is selected from the group consisting of KOAc, TEA, K₂CO₃, Na₂CO₃, and mixtures thereof. In yet another aspect of the invention, the alcohol in Steps (a), (b), and (c) is selected from the group consisting of methanol, ethanol, 1-propanol, 2-propanol and 2-butanol. In one aspect, the alcohol in Steps (a) and (b) is anhydrous and the alcohol in Step (c) is aqueous. In another aspect, the alcohol in Steps (a), (b), and (c) is ethanol. In another aspect, the co-solvent is selected from the group consisting of iPrOAc, EtOAc, DMF, DME, THF, MeTHF, acetonitrile, and mixtures thereof. In one aspect, the solutions added to the reaction in Steps (a-c) are purged with nitrogen (N₂) or argon (Ar) and the reactions in Steps (a-d) occur under an inert atmosphere of N₂ or argon (Ar).

In yet another aspect of the invention, is a process for preparing the Formula (1) compound (crude), comprising the steps of:
a) pre-activating the Formula (B) compound with the palladium catalyst Pd(OAc)₂ in the presence of anhydrous ethanol, a ligand that is XPhos, and a borylation buffer comprising the acid, HOAc, and the base, KOAc;
b) borylating the preactivated Formula (B) compound of step (a) by adding the borylating agent bisboronic acid and ethylene glycol in anhydrous ethanol;
c) mixing the Formula (A) compound with K₂CO₃, Na₂CO₃, or TEA in aqueous ethanol; and
d) mixing the reactants of Step (c) to the reactants of Step (b) to give the compound of Formula (1), and diastereomers thereof.

In yet another aspect of the invention, is a process for preparing the Formula (1) compound (crude), comprising the steps of:
a) pre-activating the Formula (B) compound with the palladium catalyst Pd(OAc)₂ in the presence of anhydrous ethanol, a ligand that is XPhos, and a borylation buffer comprising the acid, HOAc, and the base, KOAc;
b) borylating the preactivated Formula (B) compound of step (a) by adding the borylating agent bisboronic acid and ethylene glycol in anhydrous ethanol;
c) mixing the Formula (A) compound with K₂CO₃, Na₂CO₃, or TEA in aqueous ethanol; and
d) mixing the reactants of Step (c) to the reactants of Step (b) to give the compound of Formula (1), and diastereomers thereof; and wherein the solutions added to the reaction in Steps (a-c) are purged with N₂ or Ar and the reactions in Steps (a-d) occur under an inert atmosphere of N₂ or Ar.

In yet another aspect of the invention, is a process for preparing and purifying the Formula (1) compound,

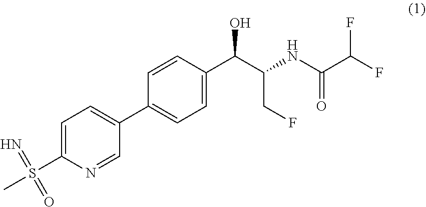 [SMILES: CS(=N)(=O)c1ccc(-c2ccc([C@@H](O)[C@@H](CF)NC(=O)C(F)F)cc2)cn1]

comprising the steps of:
a) pre-activating the Formula (B) compound with a palladium catalyst in the presence of an alcohol, a ligand, and a borylation buffer;

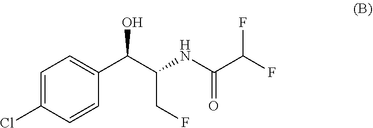 [SMILES: O=C(N[C@H](CF)[C@H](O)c1ccc(Cl)cc1)C(F)F]

b) borylating the preactivated Formula (B) compound of step (a) by adding a borylating agent in an alcohol to the reactants of Step (a);
c) mixing the Formula (A) compound with a base in an alcohol, co-solvent, or mixtures thereof;

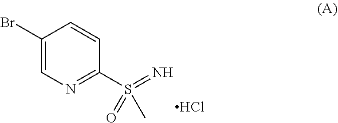

d) adding the reactants of Step (c) to the reactants of Step (b) to give the crude compound of Formula (1), and diastereomers thereof;
e) purifying the crude Formula (1) compound obtained in Step (d) by concentrating the reaction product of Step (d) and extracting the Formula (1) compound into an extracting solvent;
f) adding an aqueous wash to the extracting solvent of Step (e) while stirring; and separating the organic layer;
g) adding a palladium scavenger to the organic layer of step (f) or recycling the organic layer of step (f) with a cartridge containing a palladium scavenger, stirring and filtering out the solids, rinsing the solids with extracting solvent(s) and concentrating the filtrate;
h) dissolving the resulting concentrate of Step (g) in an organic solvent(s) with heat; cooling, and seeding the mixture with the Formula (1) compound;
i) cooling the mixture of Step (h) to about 5-25° C., optionally, add an anti-solvent; collecting the resultant solids by filtration, rinsing the solids with an anti-solvent, and then drying the solids to prepare a 1:1 diastereomeric mixture of the Formula (1) compound; the solutions added to the reaction in Steps (a-c) are purged with nitrogen (N₂) or argon (Ar) and the reactions in Steps (a-d) occur under an inert atmosphere of N₂ or argon (Ar).

In yet another aspect of the invention, is a process for preparing the Formula (1) compound, comprising the steps of:
a) pre-activating the Formula (B) compound with a palladium catalyst selected from the group consisting of Pd(OAc)₂, PdCl₂, Pd-G2-XPhos, Pd-XPhos Crotyl Cl, Pd(dppf)Cl₂, Pd-G2-PCy₃, and Pd₂(dba)₃, in the presence of an alcohol, a ligand selected from the group consisting of XPhos, SPhos, dppp, dppf, dba, PPh₃, and PCy₃, and a borylation buffer that comprises an acid and a base, wherein said acid is selected from the group consisting of HOAc, citric acid, formic acid, chloroacetic acid, and ammonium acetate;
b) borylating the preactivated Formula (B) compound of step (a) by adding a borylating agent in an alcohol wherein the borylating agent is selected from the group consisting of bisboronic acid; bisboronic acid and ethylene glycol; bisboronic acid and propylene glycol; B₂Pin₂; and B₂(NMe₂)₄;
c) mixing the Formula (A) compound with a base in an alcohol, co-solvent, or mixtures thereof;
d) mixing the reactants of Step (c) to the reactants of Step (b) to give the compound of Formula (1), and diastereomers thereof;
e) purifying the crude Formula (1) compound from Step (d) by concentrating the reaction product of Step (d) and extracting the Formula (1) compound into an extracting solvent selected from the group consisting of THF, EtOAc, MeOAc, methylene chloride and MeTHF;
f) adding an aqueous wash, selected from the group consisting of water or brine, each containing a palldium chelator selected from the group consisting of EDA, TMT-Na₃, NH₄OH, TMT, NaHSO₃, thiourea, DEA, EDTA, Ac-L-cysteine, citric acid, and mixtures thereof, while stirring and separating the organic layer;
g) adding a palladium scavenger to the organic layer from Step (f) selected from the group consisting of Carbon with EDA, silica gel with EDA, Si-Thiol, MP-TU, MP-TMT, Si-TMT, Si-DMT, and Si-cysteine and mixtures thereof; or recycling the organic layer of Step (f) through a cartridge of Carbon with EDA, silica gel with EDA, Si-Thiol, MP-TU, MP-TMT, Si-TMT, Si-DMT, and Si-cysteine while stirring, filtering out the solids, rinsing the solids with an extracting solvent(s); and concentrating the filtrate;
h) dissolving the resultant concentrate of Step (g) in an organic solvent(s) with heat, selected from the group consisting of MEK, iPrOAc, EtOAc, acetone, 1-butanol, 1-propanol, 2-propanol, and mixtures thereof, with heat >55° C., cooling to about 50-55° C. and seeding the mixture with the Formula (1) compound;
i) cooling the mixture of step (h) to about 5-25° C., optionally adding an anti-solvent selected from the group consisting of water, MTBE, hexane, heptane, and mixtures thereof; collecting the resultant solids by filtration, rinsing the solids with the anti-solvent, and then drying the solids to prepare a 1:1 diastereomeric mixture of the Formula (1) compound; wherein the solutions added to the reaction in Steps (a-c) are purged with nitrogen (N₂) or argon (Ar) and the reactions in Steps (a-d) occur under an inert atmosphere of N₂ or argon (Ar).

As described above, the base in Step (a) and (c) is selected from the group consisting of KOAc, CsOAc, TEA, K₂CO₃, Na₂CO₃, Cs₂CO₃, DIPEA, and K₃PO₄, and mixtures thereof. In yet another aspect of the invention, the base in Step (a) and (c) is selected from the group consisting of KOAc, TEA, K₂CO₃, Na₂CO₃, and mixtures thereof. In yet another aspect of the invention, the base in Step (a) and (c) is selected from the group consisting of KOAc, TEA, and K₂CO₃, and mixtures thereof. In yet another aspect of the invention, the alcohol in Steps (a), (b), and (c) is selected from the group consisting of methanol, ethanol, 1-propanol, 2-propanol and 2-butanol. In one aspect, the alcohol in Steps (a) and (b) is anhydrous and the alcohol in Step (c) is aqueous. In another aspect, the alcohol in Steps (a), (b), and (c) is ethanol. In another aspect, the co-solvent is selected from the group consisting of iPrOAc, EtOAc, DMF, DME, THF, MeTHF, acetonitrile, and mixtures thereof. In another aspect, the co-solvent is selected from the group consisting of THF, MeTHF, acetonitrile, and mixtures thereof. In another aspect, the co-solvent is selected from the group consisting of THF, MeTHF, and mixtures thereof. In another aspect, the co-solvent is THF. In one aspect, the solutions added to the reaction in Steps (a-c) are purged with nitrogen (N₂) or argon (Ar) and the reactions in Steps (a-d) occur under an inert atmosphere of N₂ or argon (Ar). In another aspect, the aqueous wash in Step (f) is a solution selected from the group consisting of water or brine, each containing a palladium chelator selected from the group consisting of EDA, TMT-Na₃, NH₄OH, TMT, NaHSO₃, thiourea, DEA, EDTA, Ac-L-cysteine, citric acid and mixtures thereof. In another aspect, the aqueous wash in Step (f) is a brine solution containing a palladium chelator selected from EDA, NH₄OH, and mixtures thereof. In another aspect, the organics in Step (f) are washed 1×, 2×, 3×, or 4× with the aqueous wash. In another aspect, the organics in Step (f) are washed 2× with the aqueous solution. In another aspect, the organics in Step (f) are washed 3× with the aqueous solution. In another aspect, the organics in Step (f) are washed 4× with the aqueous solution.

In yet another aspect of the invention, is a process for preparing the Formula (1) compound, comprising the steps of:
a) pre-activating the Formula (B) compound with the palladium catalyst, Pd(OAc)₂ in the presence anhydrous ethanol, a ligand that is XPhos, and a borylation buffer comprising an acid that is HOAc and a base that is KOAc; b) borylating the preactivated Formula (B) compound of step (a) by adding a borylating agent that is bisboronic acid and ethylene glycol in anhydrous ethanol;
c) mixing the Formula (A) compound with a K₂CO₃, Na₂CO₃, TEA, or mixtures thereof, in aqueous ethanol, or a co-solvent, or mixtures thereof;
d) mixing the reactants of Step (c) to the reactants of Step (b) to give the compound of Formula (1), and diastereomers thereof;
e) purifying the crude Formula (1) compound of Step (d) by concentrating the reaction product of Step (d) and extracting the Formula (1) compound into the extracting solvent, THF;
f) adding an aqueous wash consisting of water or brine, each of which contain a palladium chelator selected from the group consisting of EDA, TMT-Na₃, NH₄OH, TMT, NaHSO₃, thiourea, DEA, EDTA, Ac-L-cysteine, citric acid and mixtures thereof; and separating the organic layer;
g) adding a palladium scavenger selected from the group consisting of Carbon with EDA, silica gel with EDA, Si-Thiol, MP-TU, MP-TMT, Si-TMT, Si-DMT, and Si-cysteine and mixtures thereof, to the organic layer of step (f) or recycling the organic layer of step (f) through a cartridge of Carbon with EDA, silica gel with EDA, Si-Thiol, MP-TU, MP-TMT, Si-TMT, Si-DMT, and Si-cysteine, stirring and filtering out the solids, rinsing the solids with the extracting solvent(s), and concentrating the filtrate;
h) dissolving the resultant concentrate of Step (g) in an organic solvent(s) selected from the group consisting MEK, iPrOAc EtOAc, acetone, 1-butanol, 1-propanol, 2-propanol and mixtures thereof with heat >55° C., cooling to about 50-55° C., and seeding the mixture with the Formula (1) compound;
i) cooling the mixture of step (h) to about 5-25° C., optionally adding an anti-solvent selected from the group consisting of water, MTBE, hexane, heptane, and mixtures thereof, collecting the resultant solids by filtration, rinsing the solids with the anti-solvent, and then drying the solids to prepare a 1:1 diastereomeric mixture of the Formula (1) compound; wherein the solutions added to the reaction in Steps (a-c) are purged with nitrogen (N₂) or argon (Ar) and the reactions in Steps (a-d) occur under an inert atmosphere of N₂ or argon (Ar).

In yet another aspect of the invention, is a process for preparing the Formula (1) compound, comprising the steps of:
a) pre-activating the Formula (B) compound with the palladium catalyst, Pd(OAc)₂ in the presence of anhydrous ethanol, a ligand that is XPhos, and a borylation buffer comprising an acid that is HOAc and a base that is KOAc; b) borylating the preactivated Formula (B) compound of step (a) by adding a borylating agent that is bisboronic acid and ethylene glycol in anhydrous ethanol;
c) mixing the Formula (A) compound with a K₂CO₃, Na₂CO₃, TEA, or mixtures thereof, in aqueous ethanol, or a co-solvent, or mixtures thereof;
d) mixing the reactants of Step (c) to the reactants of Step (b) to give the compound of Formula (1), and diastereomers thereof; and wherein the solutions in Steps (a-c) are purged with N₂ and the reactions in Steps (a-d) occur under an inert atmosphere of N₂ or Ar;
e) purifying the crude Formula (1) compound of Step (d) by concentrating the reaction product of Step (d) and extracting the Formula (1) compound into the extracting solvent, THF;
f) adding an aqueous wash, that is a NaCl brine solution containing a palladium chelator selected from EDA, NH₄OH, and mixtures thereof, to the THF of Step (e) while stirring, and separating the organic layer;
g) adding the palladium scavenger, Carbon with EDA to the organic layer of Step (f) or recycling the organic layer of Step (f) through a Carbon cartridge with EDA, stirring and filtering out the solids, rinsing the solids with the extracting solvent, THF, and concentrating the filtrate;
h) dissolving the resultant concentrate of Step (g) in 1-propanol with heat >55° C., cooling to about 50-55° C. and seeding the mixture with the Formula (1) compound;
i) cooling the mixture of Step (h) to about 5-25° C., adding optional heptane, collecting the resultant solids by filtration, rinsing the solids with heptane, drying the solids to prepare a 1:1 diastereomeric mixture of the Formula (1) compound; wherein the solutions added to the reaction in Steps (a-c) are purged with nitrogen (N₂) or argon (Ar) and the reactions in Steps (a-d) occur under an inert atmosphere of N₂ or argon (Ar).

In yet another aspect of the invention, is a process for preparing the Formula (1) compound comprising the steps of:
a) combining Pd(OAc)₂, XPhos, the Formula (B) compound, and KOAc under a N₂ atmosphere; preparing a solution of N₂ purged, anhydrous EtOH and HOAc and adding to the palladium mixture; heating to about 72° C. for about 30 minutes and then cooling to about 52° C.;
b) borylating the Formula (B) compound by adding a N₂-purged solution of bisboronic acid and ethylene glycol in anhydrous EtOH to the mixture of Step (a) over about 30 minutes at about 52° C.;
c) mixing a N₂-purged solution of the Formula (A) compound and K₂CO₃ in aqueous THF at about 42° C.;
d) mixing the reactants of Step (b) and Step (c); then heating to about 72° C. while stirring until the coupling of Formula (A) and (B) are complete, about 4 hours;
e) cooling the mixture from Step (d) to ambient temperature, neutralizing the mixture to pH 7.0 with concentrated HCl; concentrating the reaction product, adding water and extracting the concentrate into the extracting solvent, THF, and separating the extraction layer;
f) washing the extraction of step (e) with an aqueous wash of NaCl brine with the palladium chelator, EDA, and separating the organic layer;
g) adding a palladium scavenger of Carbon with EDA to the organic layer of step (f) or recycling the organic layer of step (f) through a cartridge of Carbon with EDA while stirring, filtering out the solids, rinsing the solids with the extracting solvent, THF, and concentrating the filtrate;
h) dissolving the resultant concentrate of Step (g) with heat at about >55° C. in 1-propanol, cooling to about 55° C. and seeding with Formula (1) compound;
i) cooling the mixture from Step (h) to about 5-25° C., optionally adding the anti-solvent heptane, collecting the solids by filtration and rinsing with heptane; drying the solids in vacuo at about 57° C. to afford the 1:1 diastereomeric mixture of the Formula (1) compound.

In yet another aspect of the invention, is a veterinary composition comprising the Formula (1) compound, or a veterinary acceptable salt thereof, prepared from the process as described herein. In yet another aspect of the invention, the veterinary composition further comprises a veterinary acceptable carrier.

In yet another aspect of the invention, is a method for controlling or treating bacterial infections in an animal by administering to an animal in need of a therapeutically effective amount of the Formula (1) compound prepared from the process described herein.

In yet another aspect of the invention, is a process for preparing the Formula (A) compound comprising the oxidation of the sulfilimine compound of intermediate compound (c)

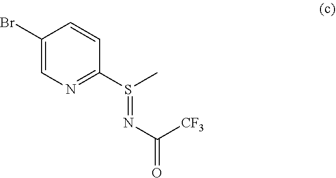 [SMILES: C/S(=N\C(=O)C(F)(F)F)c1ccc(Br)cn1]

with hydrogen peroxide and a carbonate in a solution comprising acetonitrile, an alcohol, and optionally, water. In another aspect, the alcohol is methanol. The ratio of acetonitrile to methanol is about 75:25 to 50:50. In another aspect, the solution contains water. In another aspect, the amount of acetonitrile, methanol, and water, is about 126 mL, 74 mL, and 4.3 mL, respectively. In another aspect, the carbonate is potassium carbonate.

### DETAILED DESCRIPTION

### Brief Description of the Drawings

FIG. 1. Depicts an illustrative PXRD pattern of crystalline Form A1; the diastereomreric mixture of Formula 1a and 1b [50.83:49.17; 1a/1b˜1.03).

FIG. 2. Depicts an illustrative PXRD pattern of crystalline Form A2; the diastereomreric mixture of Formula 1a and 1b [47.46:52.54; 1a/1b˜0.903].

FIG. 3. Depicts an illustrative PXRD pattern of crystalline Form A3; the diastereomreric mixture of Formula 1a and 1b [56.43:43.57; 1a/1b˜1.295].

For purposes of the present invention, as described and claimed herein, the following terms and phrases are defined as follows:

“About” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater.

“Animal” as used herein, unless otherwise indicated, refers to an individual animal, and said individual animal is a mammal. Specifically, mammal refers to a vertebrate animal that is human and non-human, which are members of the taxonomic class Mammalia. Non-exclusive examples of non-human mammals include companion animals and livestock. Preferred animals are non-human animals. Non-exclusive examples of a companion animal include: dog, cat and horse. Preferred companion animals are dog and horse. More preferred is dog. Non-exclusive examples of livestock include: sheep, goats, cattle, and swine. Preferred livestock is cattle and swine. Preferred livestock is cattle. Preferred livestock is swine.

“Seeding” or “Seeded”, as used herein, unless otherwise indicated, refers to adding the Formula (1) compound to the reaction to prepare the diastereoisomers. The Formula (1) seed can be obtained by processes previously described in U.S. Pat. No. 9,422,236; or can be prepared by processes described herein in a manner such that the original “seed” diastereomers are obtained at the end of the reaction by chromatographic separation and/or the workup followed by crystallization.

“Therapeutically effective amount”, as used herein, unless otherwise indicated, refers to an amount of the diastereomeric Formula (1) compound of the present invention that (i) treat or prevent the particular bacterial infection. A dose range of about 1 to 50 mg/kg is contemplated to be a therapeutically effective dose. A preferred dose is about 10 to 40 mg/kg. A more preferred dose is about 15 to 35 mg/kg. The most preferred dose is about 20 mg/kg.

“Treatment”, “treating”, and the like, as used herein, unless otherwise indicated, refers to reversing, alleviating, or inhibiting the bacterial infection. As used herein, these terms also encompass, depending on the condition of the animal preventing the onset of a disorder or condition, or of symptoms associated with a disorder or condition, including reducing the severity of a disorder or condition or symptoms associated therewith prior to affliction with said infection.

“Veterinary acceptable” as used herein, unless otherwise indicated, suggests that the substance or composition must be compatible chemically and/or toxicologically with the other ingredients comprising the composition and/or the animal being treated therewith. Veterinary acceptable also encompasses pharmaceutically acceptable.

In addition to the definitions described above, the following terms are defined:

“Aqueous wash” is used to wash the Formula (1) compound in the extracting solvent in the purifying step of the process for making the 1:1 diastereomeric mixture of the Formula (1) compound. Representative aqueous wash(s) is a water or brine (aqueous NaCl) solution, each containing a palladium chelator(s). Representative palladium chelators are selected from the group consisting of EDA, TMT-Na₃, NH₄OH, TMT, NaHSO₃, thiourea, DEA, EDTA, Ac-L-cysteine, citric acid and mixtures thereof. The brine solution can be saturated.

“Organic solvent” is used to dissolve the Formula (1) compound in the purifying step of the process for making the 1:1 diastereomeric mixture of the Formula (1) compound. Representative organic solvents include: MEK, iPrOAc, EtOAc, acetone, 1-butanol, 1-propanol, 2-propanol, and mixtures thereof.

“Extracting solvent” is used to extract the Formula (1) compound in the purifying steps of the process for making the 1:1 diastereomeric mixture of the Formula (1) compound. Representative extracting solvents include: THF, EtOAc, MeOAc, CH₂Cl₂, and MeTHF.

“Anti-solvent” is used to crystallize the Formula (1) compound in the purifying step of the process for making the 1:1 diastereomeric mixture of the Formula (1) compound. Representative anti-solvents include water, MTBE, hexane, heptane, and mixtures thereof.

The ratio term 1:1 as it relates to the ratio of the two diastereomers of Formula (1) (i.e., 1a:1b) refers to a ratio that is in the range from about 47:53 to about 53:47; preferably from about 48:52 to about 52:48; and more preferably from about 49:51 to about 51:49 of Formula (1a) and Formula (1b), respectively.

The compound of the instant invention contains three chiral centers. As such, certain intermediates (f and g; Scheme 2) in the preparation of Formula (B) consist of a racemic mixture of enantiomers. Each of the respective enantiomeric intermediates have identical chemical and physical properties except for their ability to rotate plane-polarized light (+/−) by equal amounts but in opposite directions. Enantiomers are also called optical isomers. A mixture of equal parts of an optically active isomer and its enantiomer has a zero-net rotation of plane-polarized light because the positive rotation of each (+) form is exactly counteracted by the negative rotation of each (−) form. To prepare the enantiomerically pure Formula (B) compound, intermediate (g) is reacted with an optically active acid (e.g., (S)-mandelic acid) to crystallize out the pure (1R,2S) biologically active (eutomer) enantiomer (intermediate h). The biologically inactive (distomer) enantiomer which stays in solution is discarded. This intermediate proceeds through further synthetic steps to provide the enantiomerically pure Formula (B). Separation of the racemic mixture of intermediate (g) can also be accomplished by standard chromatographic methods on chiral adsorbents (e.g., acetyl cellulose). Upon coupling of the racemic Formula (A) and enantiomerically pure Formula (B), the final Formula (1) compound is a mixture of diasteromers, Formula's (1a) and (1b). The mixture of diastereomers can be purified to prepare a 1:1 mixture of diasteromers.

Pharmaceutical Salts

The compound of Formula (1) may be used in its native form (base) or as a salt. The Formula (1) compound has a basic functional group and can form addition salts with acids. Such salts are included within the scope of the present invention to the extent that they are acceptable for veterinary use. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, icotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts.

Composition/Formulation

Pharmaceutical compositions of the present invention may be manufactured by processes well known in the art, e.g., by means of conventional mixing, dissolving, granulation, levigating, emulsifying, encapsulating, entrapping, lyophilizing processes or spray drying.

Pharmaceutical compositions for use in accordance with the present invention may be formulated in conventional manner using one or more pharmaceutically acceptable carriers comprising excipients and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant invention. Such excipients and carriers are described, for example, in “Remington's Pharmaceutical Sciences”, Mack Pub. Co., New Jersey (1991). In one aspect, the composition comprising the Formula (1) compound is an injectable composition. Injection can be subcutaneous, intra-muscular, by intravenous. The preferred route of injection is subcutaneous. As described by “Remington”, the composition comprises excipients and carriers that are known to provide solubility to the compound of Formula (1) and that are acceptable for pharmaceutical veterinary use. For example, an injectable composition can comprise the Formula (1) compound, DMSO and DMA. Other commonly used excipients and/or carriers can include glycerol, glycols, dialkylglycol ethers, and the like. The composition can also include an anti-oxidant (e.g., BHA, BHT, phenol, and mixtures thereof) and/or preservatives (e.g., benzyl alcohol, citric acid, and the like). The composition can comprise about 100 mg/mL to about 600 mg/mL of the Formula (1) compound per milliliter of carrier(s). A preferred composition contains about 200 mg/mL to 500 mg/mL of the Formula (1) compound. A preferred composition contains about 200 mg/mL of the Formula (1) compound. A preferred composition contains about 300 mg/mL of the Formula (1) compound. A preferred composition contains about 400 mg/mL of the Formula (1) compound. A preferred composition contains about 500 mg/mL of the Formula (1) compound. The composition can also contain amounts of the Formula (1) compound at about 250 mg/mL, 350 mg/mL, and 450 mg/mL. Other amount/volume compositions are also construed herein.

The formulations of the invention can be designed to be short-acting, fast-releasing, long-acting, extended-releasing, or controlled-releasing. Specifically, the formulation of the invention can be an extended release form. Thus, the pharmaceutical formulations can also be formulated for controlled release or for slow release. The pharmaceutical formulations comprise a compound of Formula (1), and may also comprise a pharmaceutically acceptable salt of the Formula (1) compound.

Dosage

Pharmaceutical compositions suitable for use in the present invention include compositions wherein the active ingredient i.e., the Formula (1) compound, is contained in an amount sufficient to achieve the intended purpose, i.e., control or the treatment of infections. More specifically, a therapeutically effective amount means an amount of compound effective to prevent, alleviate or ameliorate symptoms/signs of infections or prolong the survival of the subject being treated.

The quantity of active component, which is the compound of this invention, in the pharmaceutical composition and unit dosage form thereof, may be varied or adjusted widely depending upon the manner of administration, the potency of the particular compound and the desired concentration. Determination of a therapeutically effective amount is well within the capability of those skilled in the art. Generally, the quantity of active component will range between 0.01% to 99% by weight of the composition.

Generally, a therapeutically effective amount of dosage of the Formula (1) compound will be in the range of about 1 to 50 mg/kg of body weight/day; preferably about 10 to 40 mg/kg body weight/day; and more preferrably about 15 to 35 mg/kg body weight/day; and most preferably about 20 mg/kg body weight/day. It is to be understood that the dosages may vary depending upon the requirements of each subject and the severity of the bacterial infection.

The desired dose may conveniently be presented in a single dose or as divided doses administered at appropriate intervals, for example, as two, three, four or more sub-doses per day. Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

Antibacterial Assays

Compounds of the present invention are tested against an assortment of Gram-negative and Gram-positive organisms using the industrial standard techniques described in M31-A3. Performance Standards for Antimicrobial Disk and Dilution Susceptibility Tests for Bacteria Isolated from Animals; Clinical and Laboratory Standards Institute, Approved Standard-Third Edition. The compounds of the present invention demonstrate very good antibacterial activity against BRD pathogens, for example, M. haemolytica, P. multo., H. somnus and M. bovis.

Medical and Veterinary Uses

The Formula (1) compound of the present invention is an antibacterial agent that is used for the treatment of bovine respiratory disease infections in cattle caused by Gram-negative respiratory pathogens, such as M. haemolytica, P. multocida, H. somnus, and M. bovis.

In one study, healthy dairy calves (N=15/group; 85-140 kg) were trans-tracheally infected −2 to −1 days with the A3579 (OSU-012103-BHI) strain of Mannheimia haemolytica. The challenge dose ranged from about 2.9×109 CFU to 4.9×108 CFU. On Day 0, BRD symptomatic animals received a single subcutaneous dose of saline (control) or test (20 or 40 mg/kg) compound. At necropsy, control animals presented with an upper range of lung lesions of about 45%. Animals dosed with the 20 and 40 mg/kg dose presented with significantly lower percentages of lung lesion at upper range values of about 12% and 8%, respectively. Therefore, a single dose of the Formula (1) compound administered subcutaneously was effective for the treatment of BRD caused by M. haemolytica in dairy calves.

In a second study, cattle (mixed beef and/or dairy breeds; 180-270 kg) were treated from a naturally occurring BRD infection. Animals (n=40/group) received a single subcutaneous dose of saline (control) or test (15 mg/kg or 20 mg/kg) compound. Overall, treatment success, based on respiratory symptoms (rate, mucopurulent nasal or ocular discharge, open mouth breathing) and attitude (alert, stimuli response time, muscle weakness, ataxia, swaying) was about 37% and 47% for the 15 mg/kg and 20 mg/kg dose, respectively. In contrast, control animal success was about 20%. Overall, the Formula (1) compound was shown to provide significant treatment effect in cattle with natural BRD infections.

In a third study, Holstein/Holstein cross cattle approximately 6 months of age weighing about 330 kg were trans-tracheally challenged with ˜3×10⁹ CFU/dose of M. haemolytica (strain 34195). Animals were dosed with either saline (T01, negative control), Nuflor (florfenicol, T02, positive control, 40 mg/kg), Baytril (enrofloxacin, T03, positive control), Formula (1) compound (T04, 20 mg/kg), and Formula (1) compound (T05, 40 mg/kg). Doses were administered subcutaneously between 4-6 hours post challenge. By the end of the study (6 days), mortality and lung lesions were assessed. Mortality for T01, T02, T03, T04, and T05 were 53.3%, 46.7%, 0%, 0%, and 0%, respectively. Animals treated with T03, T04, and T05 showed a significant reduction in BRD related mortality compared to T01 and T02. Bback transform LSmean(1) percent lung lesions for T01, T02, T03, T04, and T05 were 35.1%, 32.1%, 9.1%, 13.3%, and 6.8%, respectively. All T03, T04, and T05 treated animals displayed a significant reduction in percent lung lesions compared with T01 and T02 reated animals. T04 and T05 were not different form T03.

### EXAMPLES

The process for making the Formula (1) compound is illustrated by the following schemes and procedural steps. The starting materials and various reactants can be obtained from commercial sources, or are readily prepared from commercially available organic compounds, using well-known methods to one skilled in the art. The following acronyms described herein are defined: sodium thiomethoxide (NaSMe); ethanol (EtOH); and 2-propanol (isopropanol, IPA, 2-PrOH); 1-propanol (n-propanol, 1-PrOH), 2-butanol (2-BuOH), isopropyl acetate (iPrOAc), ethyl acetate (EtOAc), methyl acetate (MeOAc), 2-methyl tetrahydrofuran (MeTHF), N-methyl-2-pyrrolidone (NMP); dimethylformamide (DMF), sodium hydride (NaH); methanol (MeOH); sodium carbonate (Na₂CO₃); sodium sulfite (Na₂SO₃); magnesium sulfate (MgSO₄); tetrahydrofuran (THF); brine (aqueous NaCl), thionyl chloride (SOCl₂); dimethylsulfoxide (DMSO); hydrochloric acid (HCl); bisboronic acid (BBA); bis(pinacolato)diboron (B₂Pin₂), tetrakis(dimethylamino)diboron B₂(NMe₂)₄, methyl tert-butyl ether (MTBE); benzonitrile (PhCN); methylene chloride (CH₂Cl₂); acetonitrile (MeCN, CH₃CN); dimethoxyethane (DME); dimethylacetamide (DMAc/DMA); triethylamine (TEA); diisopropylethylamine (DIPEA), hydrogen peroxide (H₂O₂); sodium methoxide (NaOMe); methyl ethyl ketone (MEK); potassium carbonate (K₂CO₃); cesium carbonate (Cs₂CO₃); cesium acetate (CsOAc), potassium borohydride (KBH₄); potassium phosphate tribasic (K₃PO₄), 1,3-dibromo-5,5-dimethylhydantoin (DBDMH); potassium tert-butoxide (KOtBu); acetic acid (HOAc); potassium acetate (KOAc); palladium (Pd); palladium(II) acetate, (Pd(OAc)₂); palladium (II) chloride (PdCl₂), 2-dicyclohexylphosphino-2′,4′,6′-triisopropylbiphenyl (X-Phos); 1,3-bis(diphenylphosphino)propane (dppp); aminobiphenyl palladium chloride precatalyst (Pd-G2-XPhos), chloro(crotyl)(2-dicyclohexylphosphino-2,4,6-triisopropylbiphenyl)palladium (II) (Pd-XPhos Crotyl Cl), tricyclohexylphosphine aminobiphenyl palladium chloride precatalyst (Pd-G2-PCy₃), bis(diphenylphospino)ferrocene dichloropalladium (Pd(dppf)Cl₂), tris(dibenzylideneacetone)dipalladium (Pd₂(dba)₃, 2,2,2-trifluoroacetamide (CF₃CONH₂); ethyldifluoroacetate (MeO₂CCHF₂); ethylene glycol (EG), propylene glycol (PG); 2-dicyclohexylphosphino-2,6-dimethoxybiphenyl (SPhos), ferrocenediyl-bis(diphenylphosphine (dppf); dibenzylideneacetone (dba); triphenylphosphine (PPh₃); tricyclohexylphosphine (PCy₃); ethylenediamine (EDA), Carbon (activated carbon), silica el (SiO₂), siliamets thiol (Si-Thiol), quadrapure TU (MP-TU), microporous polystyrene bound-trimercaptotriazine (MP-TMT), silica bound trimercaptotriazine (Si-TMT), siliamets DMT (Si-DMT), siliamets cysteine (Si-cysteine), ammonium hydroxide (NH₄OH), trimercaptotriazine (TMT), trimercaptotriazine sodium salt (TMT-Na₃), sodium bisulfite (NaHSO₃); thiourea (H₂NCSNH₂), diethylamine (DEA), ethylenediaminetetra acetic acid (EDTA), acetyl-L-cysteine (Ac-L-cysteine), aqueous (aq); volume (V); equivalent (eq); and megapascal (MPa).

Alternatively, the Formula (1) compound can be prepared by methods first described in U.S. Pat. No. 9,422,236. The method described herein has numerous advantages over the method described in U.S. Pat. No. 9,422,236. For example:
1) synthesis of Formula (A) has 2 fewer steps, eliminates column chromatography, and increases the yield by about 67%;
2) starts with cheaper chloro-core (Formula B) versus an iodo-core (Step 6, Example 17);
3) uses an unprotected chloro-core versus a protected iodo-core, allowing two steps (borylation and Suzuki) to be done in one reactor versus three-steps (borylation, Suzuki, and deprotection) being done in three reactors;
4) borylation produces a more reactive and atom efficient ethylene glycol core boronate versus the pinacol core boronate (Step 8) resulting in easy-to-remove ethylene glycol waste versus pinacol waste in the Suzuki reaction;
5) uses one workup and crystallization for a more cost efficient manufacturing process to give a 70-80% yield of white product versus 3 workups and 3 chromatographic purifications for a 41% yield of brown solid (Step 10);
6) incorporates class 3 ethanol borylation solvent versus class 2 dioxane and cheaper Pd(OAc)2 catalyst versus Pd(PPh₃)₂Cl₂;
7) uses a) lower borylation temperatures (<60° C. versus 90° C.) and shorter times (4 hours versus 22 hours); and b) lower Suzuki temperatures (<70° C. versus 80° C.) and shorter times (4 hours versus 8 hours);
8) incorporates THF extracting solvent versus DCM to improve the workup through-put and minimize the workup processing to one reactor versus two reactors;
9) uses air-stable tetrahydroxydiboron borylating reagent versus hexamethylditin to make reactive intermediate boronate for Suzuki coupling versus the organostannane intermediate (Step 2) that could possibly Stille couple with an intermediate in the reaction; and
10) uses EDA and carbon to remove residual palladium that was probably present in the original brown solid (per internal investigations, chromatographic purifications of the Formula (1) compound as previously prepared does not remove palladium to <100 ppm.

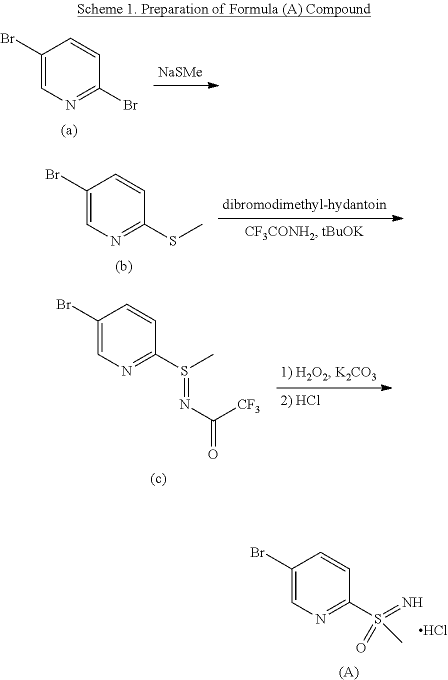

Formula (A) is prepared in 3 steps from 2,5-dibromopyridine. 2,5-dibromopyridine (a) is treated with an aqueous solution of sodium thiomethoxide in a polar, aprotic solvent such as DMF, NMP, DMAc, DMSO, preferably DMF to afford intermediate (b) which is precipitated by addition of water. The N-(trifluoroacetyl)sulfilimine (c) is synthesized by treatment of a solution of sulfide (b) and trifluoroamide with a strong base such as KOtBu, NaH, preferably KOtBu followed by dibromodimethylhydantoin. A variety of solvents can be used including MTBE, acetonitrile, THF, MeTHF, dichloromethane, and 1,4-dioxane with varying levels of the sulfoxide impurity also being formed. The sulfilimine (c) can be isolated from organic (IPA/heptane) or aqueous (alcohol/water, THF/water) based systems. Oxidation of the sulfilimine (c) to the racemic sulfoximine Formula (A) is accomplished using hydrogen peroxide, in the presence of a carbonate base, preferably powdered potassium carbonate, and a mixture of acetonitrile and a small chain alcohol, preferably methanol. The solvent combination and ratio of acetonitrile and an alcohol is critical to the success of the oxidation and control of the sulfone impurity formation. Isolation of the HCl salt of the sulfoximine compound Formula (A) purges residual sulfone and other impurities.

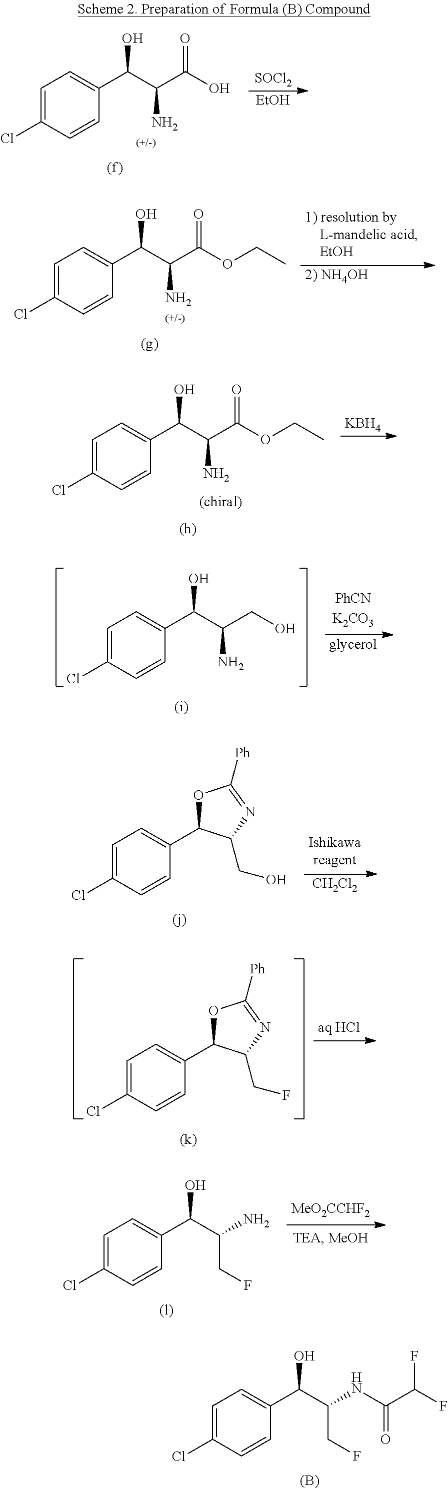

Preparation of enantiomerically pure Formula (B) is accomplished in 8 steps from 4-chlorobenzaldehyde and glycine. Reaction of glycine with 2 equivalents of 4-chlorobenzyaldehyde under basic conditions gives the coupled acid racemate intermediate (f) after neutralization. Esterification is accomplished using thionyl chloride in ethanol affording the ester racemate (g). Classical resolution of (g) is accomplished using L-mandelic acid. The ester functionality of intermediate (h) is reduced with KBH₄ and the resulting intermediate (i) protected as the dihydrooxazole without isolation using benzonitrile, glycerol and K₂CO₃ at high temperature to afford compound (j). Intermediate (j) is fluorinated using Ishikawa reagent under pressure in dichloromethane at ˜100° C. The resulting intermediate (k) is then hydrolyzed with aqueous HCl. Finally, Formula (B) compound is obtained by amidation of intermediate (I) with methyl trifluoroacetate and triethylamine. Ph in (j) and (k) is phenyl. This reaction is also described in CN106631872A.

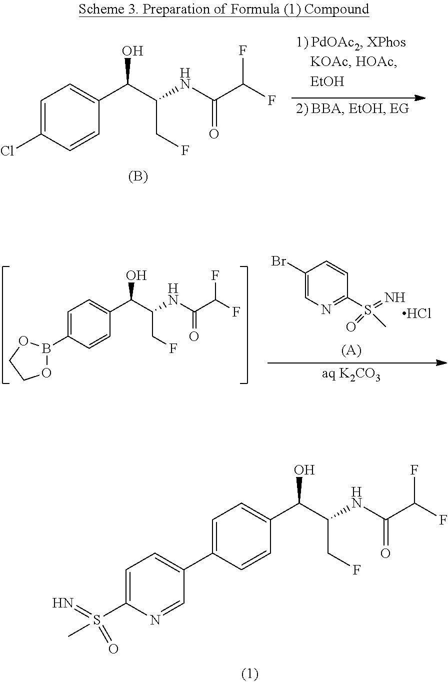

The Formula (1) compound is prepared in a 2-step, telescoped process by coupling intermediates (A) and (B). Intermediate B is borylated via a palladium catalyzed reaction using bisboronic acid. The palladium species, ligand, catalyst activation protocol, solvent system, and buffer used are important reactants in the reaction. Of particular note, is the addition of acetic acid to the reaction which improves the conversion to product compared to when acetic acid is not used. After the borylation is complete, an aqueous THF solution of Formula (A) in THF and aqueous K₂CO₃ is added. The same palladium catalyst is then used to couple the two intermediates to afford The Formula (1) compound.

### Preparation of Formula (A) Compound, (5-bromopyridin-2-yl)(imino)(methyl)-λ6-sulfanone hydrochloride

### Step-1A. Preparation of Intermediate (b), 5-bromo-2-methylsulfanyl-pyridine

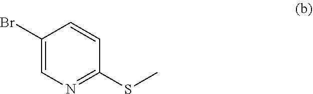

To a stirred solution of 2,5-dibromopyridine (intermediate (a) (Scheme 1); 100 g, 0.49 mol) in DMF (800 mL) was added 20% solution of sodium thiomethoxide in water (41.2 g, 0.59 mol) at about 15-20° C. The temperature was increased and held at about 50-55° C. for about 6 hours. After the reaction was complete, the batch was cooled to about 15-20° C. and water (1.8 L) was added. The batch was cooled to about 0-5° C. and after about 1-2 hours, the solid was collected by filtration, washed with water, and dried to afford the title compound as a colorless solid (75 g, 87% yield). 1H-NMR (400 MHz, DMSO) d: 2.49 (s, 3H), 7.29 (d, 1H, J=8.76 Hz), 7.85-7.88 (dd, 1H, J1=2.44 Hz, J2=8.48 Hz), 8.55 (d, 1H, J=2.4 Hz). LC-MS (m/z): M+H=206.1.

Alternatively, the compound can be made according to known methods in the art (WO2014/172443A1), or small amounts can be purchased commercially.

### Step-2A. Preparation of Intermediate (c), N-((5-bromopyridin-2-yl)(methyl)-λ4-sulfanylidene)-2,2,2-trifluoroacetamide

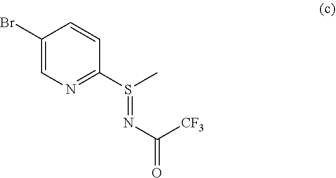 [SMILES: C/S(=N\C(=O)C(F)(F)F)c1ccc(Br)cn1]

To a solution of 5-bromo-2-methylsulfanyl-pyridine (1.5 kg, 7.35 mol) and 2,2,2-trifluoroacetamide (961 g, 1.2 eq) in MTBE (9 L) is added potassium t-butoxide (875 g, 1.10 eq) in portions keeping the batch temperature <10° C. Separately 1,3-dibromo-5,5-dimethylhydantoin (DBDMH, 2230 g, 1.10 eq) is dissolved in THF (7.2 L) and the solution cooled to about 0-5° C. The DBDMH solution is added to the batch while maintaining the batch temperature at about −5 to 10° C. After the reaction is complete, a solution of sodium sulfite in water (1200 g Na₂SO₃/5.2 L water) is added while maintaining the batch temp at <12° C. Water (6 L) is added and the batch is warmed to about 20-25° C. The layers are separated and the org layer washed with water (4.5 L) and then half saturated brine (2×4.5 L) solution. The organic layer is concentrated by vacuum distillation to a thick slurry (˜1.5 L batch volume). Isopropanol (1.5 L) is added to the batch and vacuum distillation continued to a batch volume of ˜1.5 L. A solution of 1:1 isopropanol:heptane (1.5 L) is added to the slurry and the batch cooled to about 0-5° C. The solids are collected and the cake washed with 1:2 isopropanol:heptane (1.5-2.7 L). The product is dried in vacuo at about 50° C. to give the title compound as a colorless solid (2.09 kg, 90% yield). ¹H NMR (600 MHz, DMSO-d₆) δ ppm 3.15 (s, 3H) 7.90 (d, J=8.44 Hz, 1H) 8.43 (br d, J=8.44 Hz, 1H) 8.98 (s, 1H). LC-MS (m/z): M+H=315/317.

Alternatively, to a solution of potassium t-butoxide (56.7 g, 1.02 eq) in THF (200 mL) was added a solution of 5-bromo-2-methylsulfanyl-pyridine (100 g, 0.49 mol) and 2,2,2-trifluoroacetamide (58.2 g, 1.05 eq) in THF (100 mL) while maintaining a batch temperature <10° C. Separately 1,3-dibromo-5,5-dimethylhydantoin (DBDMH, 98.1 g, 0.70 eq) is dissolved in THF (350 mL) and the solution cooled to 0-10° C. The DBDMH solution is added to the batch while maintaining the batch temperature at about ˜5 to 5° C. After the addition is complete the batch is warmed to about 10° C. When reaction is complete, a solution of sodium bisulfite (13.7 g, 0.27 eq), sodium hydroxide (13.7 g, 0.70 eq) and sodium chloride (50 g) in water (200 mL) is added and the batch is warmed to 20-25° C. Water (300 mL) is added and the layers are separated. The organic layer is concentrated by vacuum distillation to a batch volume of ˜200 mL. Isopropanol (200 mL) is added to the batch and vacuum distillation continued to a batch volume of ˜300 mL. The batch is cooled to ˜20° C. and then water (400 mL) is added over ˜30 minutes. The solids are collected by filtration and the cake washed with 1:4 isopropanol:water (200 mL). The product is dried in vacuo at about 50° C. to give the title compound as a colorless solid (134 g, 87% yield). ¹H NMR (600 MHz, DMSO-d₆) δ ppm 3.15 (s, 3H) 7.90 (d, J=8.44 Hz, 1H) 8.43 (br d, J=8.44 Hz, 1H) 8.98 (s, 1H). LC-MS (m/z): M+H=315/317.

### Step-3A. Preparation of Formula (A) Compound, (5-bromopyridin-2-yl)(imino)(methyl)-λ6-sulfanone hydrochloride

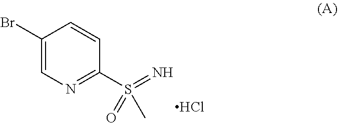

N-((5-bromopyridin-2-yl)(methyl)-A4-sulfanylidene)-2,2,2-trifluoroacetamide 50 g, 0.159 mmol) and powdered potassium carbonate (26.3 g, 1.20 eq) is combined with acetonitrile (126 mL), methanol (74 mL), and water (4.3 mL). 30% hydrogen peroxide (19.4 mL, 1.20 eq) is slowly added maintaining the batch temp at about 25-35° C. The batch is held at about 30° C. until the reaction is complete. A solution of sodium bisulfite (3.3 g, 0.2 eq) and water (150 mL) is added to the batch and stirred until no peroxide remains using a peroxide test strip. The batch is concentrated by vacuum distillation to remove the organic solvents. Dichloromethane (150 mL) is added to batch and the layers are separated. The aqueous layer is extracted with additional dichloromethane (100 mL and then 50 mL). The combined organic layer is concentrated by atmospheric distillation to a batch volume of about 250 mL. HCl in isopropanol is added to the batch keeping the batch temp at <35° C. The solids are collected at ambient temperature and washed with isopropanol (50 mL). The product is dried in vacuo at about 50° C. to give the title racemic compound as a white solid (40.4 g, 93% yield). ¹H NMR (600 MHz, methanol-d₄) δ ppm 3.95 (s, 3H) 8.30 (d, J=9.03 Hz, 1H) 8.56 (dd, J=8.28, 2.26 Hz, 1H) 9.08 (d, J=1.51 Hz, 1H). LC-MS (m/z): M+H=235/237.

The processes described above for Steps 2A and 3A provides 2 fewer steps, eliminates column chromatography, and increases the yield by about 67% compared to the previous synthesis of Formula (A) as described in U.S. Pat. No. 9,422,236.

### Process for Preparing Formula (B) Compound, N-((1R,2S)-1-(4-chlorophenyl)-3-fluoro-1-hydroxypropan-2-yl)-2,2-difluoroacetamide

### Step-1B: Preparation of Racemic Intermediate (f) (+/−) (2S,3R)-2-amino-3-(4-chlorophenyl)-3-hydroxypropanoic acid

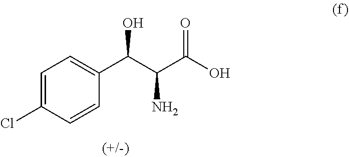 [SMILES: N[C@H](C(=O)O)[C@H](O)c1ccc(Cl)cc1]

To a solution of 4-chlorobenzyaldehyde (1152 g, 2 eq) in methanol (8 L-10 L) was added glycine (300 g) followed by 30% NaOMe in methanol (1439 g, 2 eq) and stirred at ambient temperature overnight. Aqueous HCl (810 g, 2 eq) is added and the batch stirred for 1 hour. After cooling to about 5-10° C., the product is collected by filtration, washed with ethanol and dried in vacuo at about 60° C. to afford the title compound (1.00 kg, assay 66%, 77% yield).

### Step-2B. Preparation of Racemic Intermediate (g) (+/−) ethyl (2S,3R)-2-amino-3-(4-chlorophenyl)-3-hydroxypropanoate

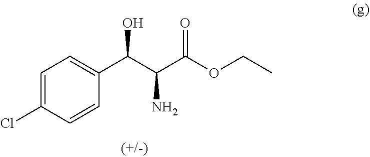 [SMILES: CCOC(=O)[C@@H](N)[C@H](O)c1ccc(Cl)cc1]

(+/−) (2S,3R)-2-amino-3-(4-chlorophenyl)-3-hydroxypropanoic acid (1.00 kg) and ethanol (4 L) are combined and cooled to <5° C. Thionyl chloride (740 g, 2 eq) is added at about 0-5° C., and then the batch is warmed to about 50° C. After the reaction is complete (10 hours), the batch is concentrated by vacuum distillation, diluted with water (1.5 L) and the pH adjusted to 8 with ammonium hydroxide. The batch is cooled to about 5-10° C. to precipitate the product which is collected by filtration, washed with water and dried to afford the titled intermediate compound (725 g, 97% yield).

### Step-3B. Chiral Resolution to Prepare Intermediate (h), ethyl (2S,3R)-2-amino-3-(4-chlorophenyl)-3-hydroxypropanoate

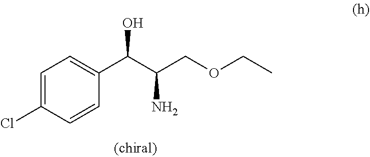

(+/−) ethyl (2S,3R)-2-amino-3-(4-chlorophenyl)-3-hydroxypropanoate (1100 g, 1 eq) is added to a solution of L-mandelic acid (1.05 eq) in ethanol (7 L) and heated to about 35-40° C. The batch is cooled to about 20° C. over 1 hour and stirred 1 hour before the solids are collected by filtration. The cake is dissolved in water (7 L) and the pH adjusted to 8-9 with ammonium hydroxide. The product is isolated by filtration, washed with water and dried in vacuo at about 45° C. to afford compound (h) (320 g, 29% yield). A second crop was obtained by concentrating the mother liquor to remove 4 L of ethanol and then cooling to about 20° C. The solids were collected by filtration and then combined with L-mandelic acid (10 g) in 1200 mL ethanol and heated to about 40° C. After cooling to about 20° C. over 1 hour and holding for 1 hour, the solids were collected by filtration and dried in vacuo at about 45° C. to afford intermediate (h) (40 g, 3.6% yield).

### Step-4B. Preparation of Intermediate (i), (1R,2R)-2-amino-1-(4-chlorophenyl)propane-1,3-diol

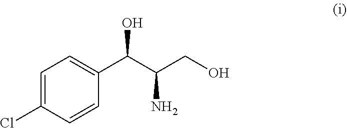 [SMILES: N[C@H](CO)[C@H](O)c1ccc(Cl)cc1]

50 g of ethyl (2S,3R)-2-amino-3-(4-chlorophenyl)-3-hydroxypropanoate was dissolved in 350 mL of methanol, and then 13.3 g of potassium borohydride was slowly added and reacted at about 40° C. for 6 hours. A suitable amount of dilute hydrochloric acid was added and stirred for 30 minutes. The solvent was evaporated off under reduced pressure. The residue was dissolved in water, adjusted to pH>10 with 30% sodium hydroxide solution, and then extracted several times with dichloromethane. The organic phases were combined, washed once with saturated brine, dried over anhydrous sodium sulfate, and filtered. The filtrate was concentrated under reduced pressure to obtain 39.7 g of a white solid (yield 96%). The crude product (i) was directly used in the preparation of a compound of Formula (j) in the next step without purification.

### Step-5B. Preparation of Intermediate (j), ((4R,5R)-5-(4-chlorophenyl)-2-phenyl-4,5-dihydrooxazol-4-yl)methanol

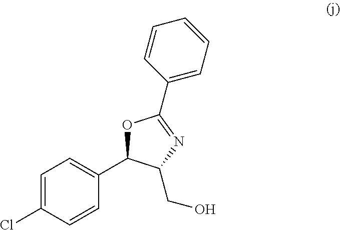 [SMILES: OC[C@H]1N=C(c2ccccc2)O[C@@H]1c1ccc(Cl)cc1]

30 g of (1R,2R)-2-amino-1-(4-chlorophenyl)propane-1,3-diol (i), 90 g of glycerin, and 6.8 g of potassium carbonate were heated to about 105° C., and then 21.5 g of benzonitrile was added dropwise in 20 minutes and reacted at about 105° C. for 18 hours. After cooling to about 50° C., 90 g of water was added, stirred at about 50° C. for 30 minutes, and then filtered while still hot. The filter cake was slurried once in ethanol, and then filtered to obtain 41.1 g of a white solid (yield 96%) of intermediate (j).

50 g of intermediate (h) is dissolved in 350 mL of methanol, and then 13.3 g of potassium borohydride is slowly added and reacted at about 40° C. for 6 hours. 150 g of glycerin is added, methanol was evaporated off by concentration under reduced pressure, and then 11.3 g of potassium carbonate is added. After the temperature was raised to about 105° C., 33.8 g of benzonitrile is added dropwise in 20 minutes, and then reacted at 105° C. for 18 hours. After cooling to about 50° C., 150 g of water is added, stirred at about 50° C. for 30 minutes, and then filtered while still hot. The filter cake is slurried once in ethanol, and then filtered to obtain 51.4 g of intermediate (j) as a white solid (yield 87%).

### Step-6B. Preparation of Intermediate (k) (4S,5R)-5-(4-chlorophenyl)-4-(fluoromethyl)-2-phenyl-4,5-dihydrooxazole

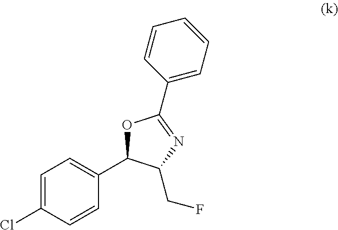 [SMILES: FC[C@H]1N=C(c2ccccc2)O[C@@H]1c1ccc(Cl)cc1]

30 g of ((4R,5R)-5-(4-chlorophenyl)-2-phenyl-4,5-dihydrooxazol-4-yl)methanol was mixed with 300 mL of dichloromethane and stirred. 24.7 mL (0.136 mol) of the Ishikawa reagent was added dropwise at room temperature under a nitrogen atmosphere, stirred until uniform and then transferred to a high-pressure reactor in which the reaction pressure was 0.6 Mpa. After reaction at about 100° C. for 2-3 hours, the reaction was cooled to room temperature, and the reaction liquid was removed. The organic phase was washed with water, adjusted to pH 6-8 with 30% sodium hydroxide solution, then washed with water again, dried over anhydrous sodium sulfate, and filtered. The filtrate was concentrated under reduced pressure and cooled to obtain a light yellow solid (k). The crude product (k) may be directly used in the next reaction without purification to prepare the compound of Formula (I).

### Step-7B. Preparation of Intermediate (I), (1R,2S)-2-amino-1-(4-chlorophenyl)-3-fluoropropan-1-ol

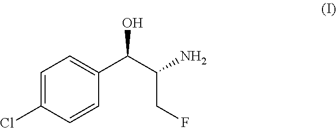 [SMILES: N[C@H](CF)[C@H](O)c1ccc(Cl)cc1]

Crude product (4S,5R)-5-(4-chlorophenyl)-4-(fluoromethyl)-2-phenyl-4,5-dihydrooxazole was added to 300 mL of 6N hydrochloric acid, heated to about 100-105° C., and reacted for 16 hours under reflux. After cooling to room temperature, the by-product benzoic acid was filtered off. The filtrate was concentrated under reduced pressure to obtain a light yellow solid, which was dissolved in water, adjusted to pH>12 with 30% sodium hydroxide solution, and then extracted twice with dichloromethane. The organic phases were combined, washed once with saturated brine, dried over anhydrous sodium sulfate, and filtered. The solvent in the filtrate was evaporated off under reduced pressure, and recrystallized in isopropanol and n-hexane to obtain 17 g of a white solid (yield 80%). Alternatively, all crude product of (k) was added to 300 mL of 6N sulfuric acid, heated to about 100-105° C., and reacted for 16 hours under reflux. After cooling to room temperature, the by-product benzoic acid was filtered off. The filtrate was concentrated under reduced pressure to obtain a light yellow solid, which was dissolved in water, adjusted to pH>12 with 30% sodium hydroxide solution, and then extracted twice with dichloromethane. The organic phases were combined, washed once with saturated brine, dried over anhydrous sodium sulfate, and filtered. The solvent in the filtrate was evaporated off under reduced pressure, recrystallized in isopropanol and n-hexane to obtain 16.7 g of a white solid (yield 78.5%).

### Step-8B. Preparation of Formula (B) Compound, N-((1R,2S)-1-(4-chlorophenyl)-3-fluoro-1-hydroxypropan-2-yl)-2,2-difluoroacetamide

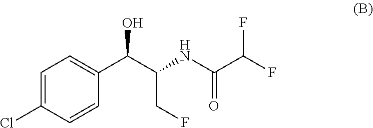 [SMILES: O=C(N[C@H](CF)[C@H](O)c1ccc(Cl)cc1)C(F)F]

10 g of (1R,2S)-2-amino-1-(4-chlorophenyl)-3-fluoropropan-1-ol was dissolved in 350 mL methanol, and then 5 g of triethyl amine and 30.5 g of ethyl difluoroacetate were added and stirred for 12 hours at room temperature. The solvent was evaporated off under reduced pressure, and the residue was recrystallized in isopropanol and water to obtain 12.7 g of a white solid (yield 92%).

### Process for Preparing the Formula (1) Compound: 2,2-difluoro-N-((1R,2S)-3-fluoro-1-hydroxy-1-(4-(6-(S-methylsulfonimidoyl)pyridin-3-yl)phenyl)propan-2-yl)acetamide

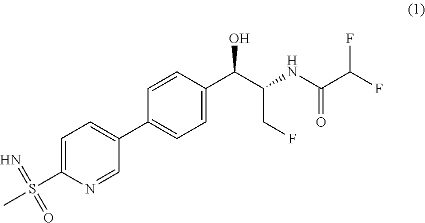 [SMILES: CS(=N)(=O)c1ccc(-c2ccc([C@@H](O)[C@@H](CF)NC(=O)C(F)F)cc2)cn1]

Under an N₂ atmosphere is combined Pd(OAc)₂ (9 g, 1 mol %) XPhos (38.9 g, 2 mol %), Formula (B) compound (1023 g, 1 eq) and KOAc (891 g, 2.5 eq). N₂ purged, 200 proof anhydrous EtOH (5115 ml) and HOAc (109 g, 0.5 eq) are added and the batch heated to about 72° C. for about 30 minutes and then cooled to about 52° C. A N₂-purged solution of tetrahydroxydiboron (bisboronic acid, BBA, 420 g, 1.25 eq) and ethylene glycol (EG, 564 g, 2.5 eq) in 200 proof anhydrous EtOH (3070 mL) is added over about 30 minutes at about 52° C. and rinsed with EtOH (450 mL). After the borylation is complete by HPLC (≤1% Formula (B), 2 hours), a N₂-purged solution of Formula (A) compound (957 g, 0.97 eq), in THF (3069 mL) and K₂CO₃ (1258 g, 2.5 eq) in water (2046 mL) at about 42° C. is added to the batch at about 52° C. and rinsed with THF:water mixture (400 mL:50 mL). The batch is heated to about 72° C. and stirred until the Suzuki coupling is complete (˜4 hours). After cooling to ambient temperature, the batch is neutralized to pH ˜7.0 with concentrated HCl. The batch is concentrated via vacuum distillation to ˜7V (batch temperature <40° C., 60-70 mBar). THF (7.2 L) is added and the batch reconcentrated via vacuum distillation (batch temp ≤30° C., 60-70 mbar) to ˜5V. THF (5.2 L) and water (3.1 L) is added and the batch at pH ˜7 is heated to about 60° C. until a biphasic solution is obtained and then cooled to 45-55° C. The bottom aqueous layer is cut away and the organic layer is stirred with water:saturated brine:EDA (1.3 L:1.3 L:0.6 eq) at ˜25 C for >0.5 hours. The lower aqueous layer is cut away. The organic layer is stirred with EDA (0.6 eq) and Carbon (660 g, activated carbon, C-941 type) for >12 hours or recycled with EDA (0.6 eq) through a cartridge(s) of Carbon (C-941 type) for >12 hours. The Carbon is filtered out and the solids rinsed with THF (2.1 L). The filtrate is washed with brine (1.3 L) with pH adjusted to ˜7 with concentrated HCl. The filtrate is washed with brine (1.3 L) and the organic layer is concentrated via vacuum distillation (batch temp ≤30° C., 60-70 mbar) to ˜2V. 1-Propanol (1-PrOH, 2.1 L) is added and the batch concentrated via vacuum distillation to ˜2V. 1-propanol (7.2 L) is added and the batch heated to about >55° C. to give a solution and then cooled to 50-55° C. and seeded. The batch is cooled to 5-25° C. and sampled. If the chiral HPLC of the solid is a 49-51% diastereomeric mixture, proceed to filtration. If the chiral HPLC of the solid is a <49% to >51% diastereomeric mixture, optional heptane (1 L) is added and the batch cooled to about 0-10° C. The solid is collected by filtration and rinsed with heptane (3V). The product is dried in vacuo at about 57° C. to afford compound (1) (70-80% yield, HPLC ≥97% area, >97% w/w assay, 49-51% ratio of diastereomers). NMR (600 MHz, DMSO) δ: 3.20 (s, 3H), 4.40 (m, 2H), 4.50 (bs, 1H), 4.60 (dq, 1H), 4.92 (bs, 1H), 5.99 (bs, 1H), 6.22 (t, 1H), 7.52 (d, 2H), 7.81 (d, 2H), 8.13 (dd, 1H), 8.39 (dd, 1H), 8.90 (d, 1H), 9.05 (d, 1H). LC-MS (m/z): M+H=402.1.

As described, the Formula 1 compound is a diastereomeric mixture of 2,2-difluoro-N-((1R,2S)-3-fluoro-1-hydroxy-1-(4-(6-((S)—S-methylsulfonimidoyl)pyridin-3-yl)phenyl)propan-2-yl)acetamide (1a) and 2,2-difluoro-N-((1R,2S)-3-fluoro-1-hydroxy-1-(4-(6-((R)—S-methylsulfonimidoyl)pyridin-3-yl)phenyl)propan-2-yl)acetamide (1b)

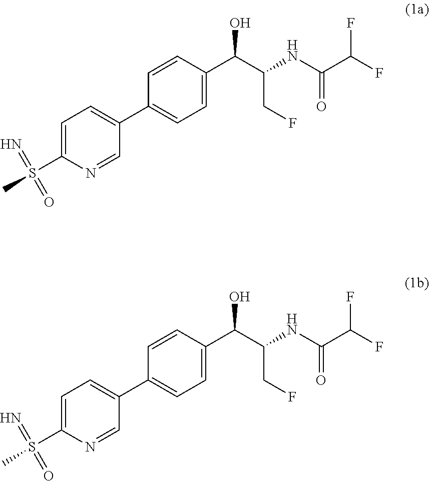 [SMILES: C[S@](=N)(=O)c1ccc(-c2ccc([C@@H](O)[C@@H](CF)NC(=O)C(F)F)cc2)cn1 ; C[S@@](=N)(=O)c1ccc(-c2ccc([C@@H](O)[C@@H](CF)NC(=O)C(F)F)cc2)cn1]

Preferrably, the diastereomers (Formula 1a and 1b) are prepared in a diastereomeric mixture in a ratio of about 48:52 (1a:1b) to 52:48 (1a:1b); and preferably from about 49:51 (1a:1b) to 51:49 (1a:1b).

Different solid-state forms of a pharmaceutical or veterinary compound can have materially different physical properties. Such differences in physical properties can have an impact, for example, on how a pharmaceutical or veterinary compound is made, processed, formulated or administered. For example, the crystalline form of one compound may have very different properties: solubility, rate of dissolution, suspension stability, stability during grinding, vapor pressure, optical and mechanical properties, hygroscopicity, crystal size, filtration properties, desiccation, density, melting point, degradation stability, stability against phase transformation into other crystalline forms, color, and even chemical reactivity. In a preferred aspect, the invention provides a specific crystalline form, preferably Form A1, of the diastereomeric mixture of the Formula (1) compound.

Crystal X-Ray Analysis

The crystal structure, as described herein, was analyzed using powder X-ray diffraction (PXRD). The X-ray diffractograms were obtained using a Bruker D4 Endeavor equipped with a LynxEye detector operated with a fixed slit and a Cu source operated at 40 kV and 40 mA, K2a wavelength 1.5406 angstroms. The diffractograms were obtained in locked coupled mode, from 5 to 50 degrees two-theta. The step size was 0.020 degrees two-theta, and the acquisition time per step was 0.5 seconds or 1 second. The divergent slit was set at 1.00 degree. A suitable blank diffractogram was subtracted. Zero background holders were employed in all tests, with the sample distributed across the surface in a thin flat layer. All tests were performed at controlled room temperature and humidity (typically 21-22° C., 25-50% RH). During acquisition, the sample holder was rotated at 20 rpm. Data were analyzed in the EVA software package obtained from Bruker.

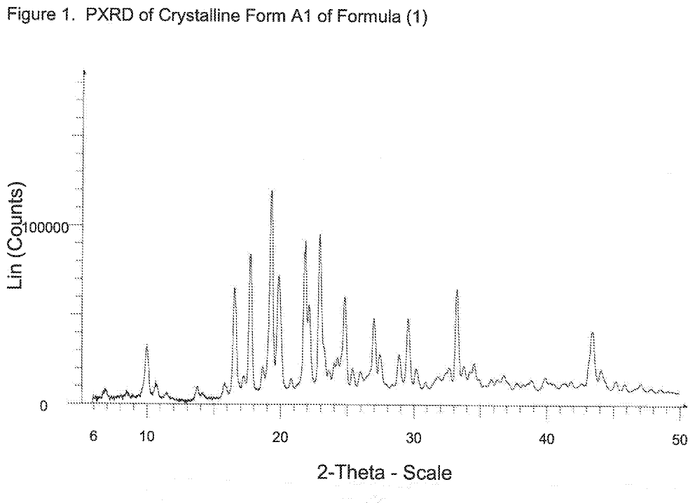

As will be appreciated by the skilled crystallographer, the relative intensities of the various peaks reported in Tables 1-3 and FIGS. 1-3, respectively, herein may vary due to a number of factors such as orientation effects of crystals in the X-ray beam or the purity of the material being analyzed or the degree of crystallinity of the sample. The PXRD peak positions may also shift for variations in sample height but the peak positions will remain substantially as defined. The skilled crystallographer also will appreciate that measurements using a different wavelength will result in different shifts according to the Bragg equation—nA, =2ci sin ⊖. Such further PXRD patterns generated by use of alternative wavelengths are considered to be alternative representations of the PXRD patterns of the crystalline materials of the present invention and as such are within the scope of the present invention. Similarly, changes in the ratio of each of the diastereomers will also affect peak intensity and potentially, peak location, when the ratio between the two diastereomers gets further from a 1:1 ratio, as depicted in Table 4.

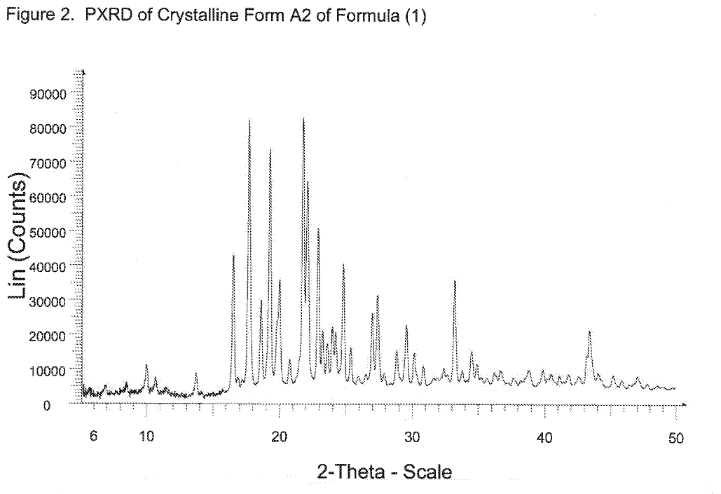

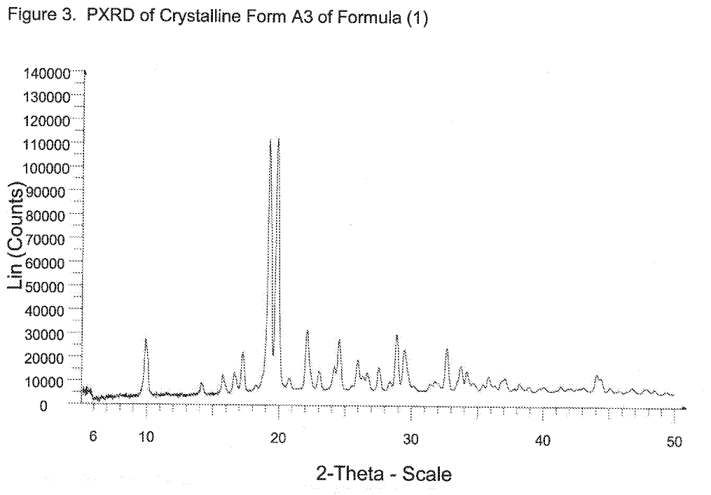

The diastereomeric (Formula 1a and 1b) mixture has a unique three-dimensional crystalline configuration that can be characterized by, inter alia, the way the crystal lattice diffracts electromagnetic radiation (e.g., PXRD). Form A1 (50.83 of 1a: 49.17 of 1b) represents the diastereomeric mixture of the Formula (1a) and (1b) compounds in a ratio of 1:1; which exhibits a PXRD pattern substantially as shown in FIG. 1. The characteristic peaks of Form A1 expressed in degrees 28 [2-Theta° ] (±0.2°), interplanar spacings (d-spacing), and respective intensities (%) are shown in Table 1. The respective PXRD peak pattern and peak characteristics of Form A2 (47.46 of 1a: 52.54 of 1b) are shown in FIG. 2 and Table 2, respectively. The respective PXRD peak pattern and peak characteristics of Form A3 (56.43 of 1a: 43.57 of 1b) are shown in FIG. 3 and Table 3, respectively. Comparative 2-theta° PXRD peaks with intensities 25% are shown in Table 4. As can be observed in the Tables and Figures, PXRD peak pattern and intensity changes within Form A depending on the relative amounts of Formula 1a and 1b. Forms A1 and A2 have similar PXRD characteristics, accounting for about 76% relative homology. Table 4 shows similar peak patterns with peak intensities 25% for Form A1, A2, and A3 at about 19.24, 19.86, and 22.09 2-Theta°. As described above, the ratio of diastereomers in the product sample provide somewhat different PXRD peak patterns and intensities, as expected.

TABLE 1
PXRD Peak Characterisitcs of Crystalline Form A1 of Formula (1)

| Peak | 2-Theta° | d-spacing | Intensity (%) |
|---|---|---|---|
| 1 | 9.92 | 8.90531 | 27.4 |
| 2 | 10.63 | 8.31471 | 9 |
| 3 | 16.52 | 5.36280 | 54.4 |
| 4 | 17.20 | 5.15009 | 13 |
| 5 | 17.69 | 5.00853 | 70.2 |
| 6 | 18.63 | 4.75846 | 17.1 |
| 7 | 19.26 | 4.60506 | 100 |
| 8 | 19.82 | 4.47593 | 60.4 |
| 9 | 20.78 | 4.27186 | 11.8 |
| 10 | 21.78 | 4.07676 | 74.3 |
| 11 | 22.09 | 4.02056 | 46.3 |
| 12 | 22.89 | 3.88242 | 79.6 |
| 13 | 23.28 | 3.81775 | 25.4 |
| 14 | 23.62 | 3.76386 | 15.9 |
| 15 | 24.00 | 3.70477 | 19 |
| 16 | 24.24 | 3.66876 | 21.5 |
| 17 | 24.77 | 3.59118 | 50 |
| 18 | 25.37 | 3.50772 | 16.5 |
| 19 | 25.97 | 3.42775 | 15.3 |
| 20 | 26.97 | 3.30312 | 40.3 |
| 21 | 27.43 | 3.24853 | 23.4 |
| 22 | 28.87 | 3.09052 | 23.4 |
| 23 | 29.55 | 3.02081 | 40.1 |
| 24 | 30.16 | 2.96036 | 16.8 |
| 25 | 31.82 | 2.81037 | 13 |
| 26 | 32.39 | 2.76168 | 14.6 |
| 27 | 32.66 | 2.73954 | 17.4 |
| 28 | 33.22 | 2.69455 | 54 |
| 29 | 33.77 | 2.65214 | 17.9 |
| 30 | 34.24 | 2.61684 | 15.5 |
| 31 | 34.54 | 2.59463 | 19.2 |
| 32 | 43.44 | 2.08173 | 34.3 |
| 33 | 44.10 | 2.05192 | 16.6 |

TABLE 2
PXRD Peak Characteristics of Crystalline Form A2 of Formula (1)

| Peak | 2-Theta° | d-spacing | Intensity (%) |
|---|---|---|---|
| 1 | 9.94 | 8.89115 | 13.4 |
| 2 | 10.62 | 8.32300 | 9 |
| 3 | 13.70 | 6.46047 | 10.4 |
| 4 | 16.49 | 5.37221 | 52 |
| 5 | 17.67 | 5.01631 | 99.5 |
| 6 | 18.59 | 4.76978 | 36 |
| 7 | 19.24 | 4.60958 | 89 |
| 8 | 19.95 | 4.44739 | 43.2 |
| 9 | 20.75 | 4.27833 | 15.4 |
| 10 | 21.74 | 4.08449 | 100 |
| 11 | 22.07 | 4.02517 | 77.6 |
| 12 | 22.87 | 3.88565 | 61.6 |
| 13 | 23.21 | 3.82967 | 25.3 |
| 14 | 23.57 | 3.77237 | 20.9 |
| 15 | 23.95 | 3.71202 | 26.5 |
| 16 | 24.20 | 3.67424 | 25 |
| 17 | 24.77 | 3.59223 | 48.9 |
| 18 | 25.34 | 3.51180 | 19.4 |
| 19 | 26.50 | 3.36135 | 10 |
| 20 | 26.97 | 3.30305 | 31.7 |
| 21 | 27.36 | 3.25697 | 37.8 |
| 22 | 27.86 | 3.19987 | 10.5 |
| 23 | 28.80 | 3.09718 | 18.6 |
| 24 | 29.54 | 3.02190 | 27.2 |
| 25 | 30.13 | 2.96419 | 17.6 |
| 26 | 30.83 | 2.89837 | 13.2 |
| 27 | 32.37 | 2.76374 | 12.4 |
| 28 | 32.65 | 2.74049 | 10 |
| 29 | 33.20 | 2.69622 | 43.1 |
| 30 | 33.78 | 2.65107 | 11.6 |
| 31 | 34.50 | 2.59768 | 18.2 |
| 32 | 34.90 | 2.56866 | 13.8 |
| 33 | 36.20 | 2.47935 | 10.9 |
| 34 | 36.70 | 2.44688 | 11.5 |
| 35 | 38.83 | 2.31720 | 11.7 |
| 36 | 39.88 | 2.25861 | 11.9 |
| 37 | 40.51 | 2.22479 | 10.3 |
| 38 | 41.82 | 2.15835 | 10.3 |
| 39 | 43.18 | 2.09334 | 16.8 |
| 40 | 43.42 | 2.08257 | 25.6 |
| 41 | 44.07 | 2.05338 | 10.7 |

TABLE 3
PXRD Peak Characteristics of Crystalline Form A3 of Formula (1)

| Peak | 2-Theta° | d-spacing | Intensity (%) |
|---|---|---|---|
| 1 | 9.90 | 8.92914 | 24.3 |
| 2 | 14.12 | 6.26703 | 7.8 |
| 3 | 15.74 | 5.62725 | 11 |
| 4 | 16.61 | 5.33439 | 11.9 |
| 5 | 17.23 | 5.14191 | 19.4 |
| 6 | 19.22 | 4.61367 | 99.6 |
| 7 | 19.82 | 4.47535 | 100 |
| 8 | 20.78 | 4.27162 | 10.1 |
| 9 | 22.13 | 4.01329 | 27.9 |
| 10 | 23.04 | 3.85702 | 12.8 |
| 11 | 24.18 | 3.67733 | 14.5 |
| 12 | 24.56 | 3.62163 | 24.5 |
| 13 | 25.98 | 3.42666 | 16.9 |
| 14 | 26.69 | 3.33770 | 12.2 |
| 15 | 27.58 | 3.23142 | 14.1 |
| 16 | 28.91 | 3.08604 | 26.6 |
| 17 | 29.50 | 3.02523 | 20.9 |
| 18 | 32.69 | 2.73699 | 21.5 |
| 19 | 33.75 | 2.65330 | 14.8 |
| 20 | 34.20 | 2.62003 | 12.8 |
| 21 | 35.85 | 2.50284 | 11 |
| 22 | 37.09 | 2.42188 | 10.4 |
| 23 | 44.11 | 2.05154 | 12.1 |
| 24 | 44.41 | 2.03844 | 10.6 |

TABLE 4
Comparative Form A PXRD 2-Theta° Peaks with Intensities ≥25%
for Form A1, A2, and A3 of Formula (1)

| Form A1 | Form A2 | Form A3 |
|---|---|---|
| 9.92 | | |
| 16.52 | 16.49 | |
| 17.69 | 17.67 | |
| | 18.59 | |
| 19.26 | 19.24 | 19.22 |
| 19.82 | 19.95 | 19.82 |
| 21.78 | 21.74 | |
| 22.09 | 22.07 | 22.13 |
| 22.89 | 22.87 | |
| 23.28 | 23.21 | |
| | 23.95 | |
| | 24.20 | |
| 24.77 | 24.77 | |
| 26.97 | 26.97 | |
| | 27.36 | |
| | | 28.91 |
| 29.55 | 29.54 | |
| | 33.20 | |
| 43.44 | 43.42 | |

1. A process for preparing the Formula (1) compound, 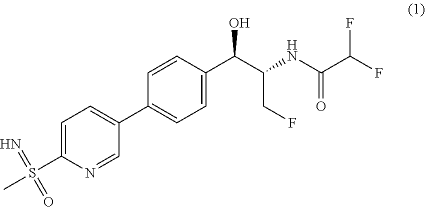 [SMILES: CS(=N)(=O)c1ccc(-c2ccc([C@@H](O)[C@@H](CF)NC(=O)C(F)F)cc2)cn1]
comprising the steps of:
a) pre-activating the Formula (B) compound with a palladium catalyst in the presence of an alcohol, a ligand, and a borylation buffer; 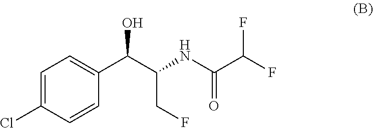 [SMILES: O=C(N[C@H](CF)[C@H](O)c1ccc(Cl)cc1)C(F)F]
b) borylating the preactivated Formula (B) compound of Step (a) by adding a borylating agent in an alcohol;
c) mixing the Formula (A) compound with a base in an alcohol, co-solvent, or mixtures thereof; and 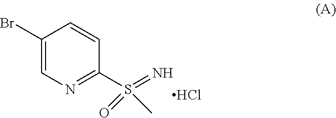
d) adding the reactants of Step (c) to the reactants of Step (b) to give the compound of Formula (1), and diastereomers thereof.

2. The process of claim 1 comprising the steps of:
a) pre-activating the Formula (B) compound with a palladium catalyst selected from the group consisting of Pd(OAc)₂, PdCl₂, Pd-G2-XPhos, Pd-XPhos Crotyl Cl, Pd(dppf)Cl₂, Pd-G2-PCy₃, and Pd₂(dba)₃, in the presence of an alcohol, a ligand selected from the group consisting of XPhos, SPhos, dppp, dppf, dba, PPh₃, and PCy₃, and a borylation buffer that comprises an acid and a base, wherein said acid is selected from the group consisting of HOAc, citric acid, formic acid, chloroacetic acid, and ammonium acetate;
b) borylating the preactivated Formula (B) compound of step (a) by adding a borylating agent in an alcohol; wherein the borylating agent is selected from the group consisting of bisboronic acid, bisboronic acid and ethylene glycol, bisboronic acid and propylene glycol, B₂Pin₂, and B₂(NMe₂)₄;
c) mixing the Formula (A) compound with a base in an alcohol, co-solvent, or mixtures thereof; and
d) mixing the reactants of Step (c) to the reactants of Step (b) to give the compound of Formula (1), and diastereomers thereof.

3. The process of claim 1, wherein the base in Step (a) and (c) is selected from the group consisting of KOAc, CsOAc, TEA, K₂CO₃, Na₂CO₃, Cs₂CO₃, DIPEA, and K₃PO₄, and mixtures thereof; and wherein the alcohol in Steps (a), (b), and (c) is selected from the group consisting of methanol, ethanol, 1-propanol, 2-propanol and 2-butanol.

4. The process of claim 3, wherein the alcohol in Steps (a) and (b) is anhydrous ethanol and the alcohol in Step (c) is aqueous ethanol.

5. The process of claim 1, wherein the co-solvent of Step (c) is selected from the group consisting of iPrOAc, EtOAc, DMF, DME, THF, MeTHF, acetonitrile, and mixtures thereof.

6. The process of claim 1 comprising the steps of:
a) pre-activating the Formula (B) compound with a palladium catalyst that is Pd(OAc)₂ in the presence of anhydrous ethanol, a ligand that is XPhos, and a borylation buffer comprising the acid, HOAc, and the base, KOAc;
b) borylating the preactivated Formula (B) compound of Step (a) by adding the borylating agent bisboronic acid and ethylene glycol in anhydrous ethanol;
c) mixing the Formula (A) compound with a K₂CO₃ or Na₂CO₃ in aqueous THF; and
d) mixing the reactants of Step (c) to the reactants of Step (b) to give the compound of Formula (1), and diastereomers thereof; and wherein solutions added to the reaction in Steps (a-c) are purged with N₂ or Ar and the reactions in Steps (a-d) occur under an inert atmosphere of N₂ or Ar.

7. A process for preparing the Formula (1) compound, 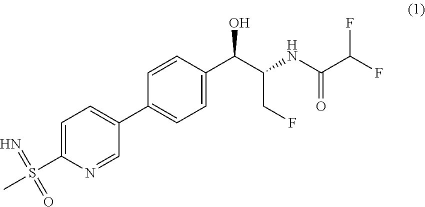 [SMILES: CS(=N)(=O)c1ccc(-c2ccc([C@@H](O)[C@@H](CF)NC(=O)C(F)F)cc2)cn1]
comprising the steps of:
a) pre-activating the Formula (B) compound with a palladium catalyst in the presence of an alcohol, ligand, and a borylation buffer; 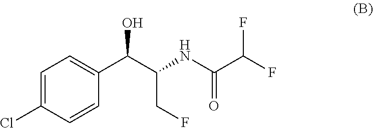 [SMILES: O=C(N[C@H](CF)[C@H](O)c1ccc(Cl)cc1)C(F)F]
b) borylating the preactivated Formula (B) compound of Step (a) by adding a borylating agent in an alcohol to the reactants of Step (a);
c) mixing the Formula (A) compound with a base in an alcohol, co-solvent, or mixtures thereof; 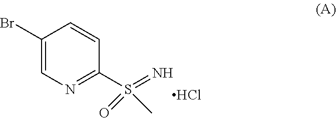
d) adding the reactants of Step (c) to the reactants of Step (b) to give the Formula (1) compound, and diastereomers thereof;
e) purifying the Formula (1) compound of Step (d) by concentrating the reaction product of Step (d) and extracting the Formula (1) compound into an extracting solvent;
f) adding an aqueous wash to the extracting solvent of Step (e), stirring the extraction and wash and separating the organic layer;
g) adding a palladium scavenger to the organic layer of Step (f) or recycling the organic layer of step (f) through a cartridge of metal scavenger, stirring and filtering out the solids, rinsing the solids with an extraction solvent(s) and concentrating the filtrate;
h) dissolving the resultant concentrate of Step (g) in an organic solvent(s) with heat, cooling and seeding the mixture with the Formula (1) compound;
i) cooling the mixture of step (h), adding an optional anti-solvent, collecting the resultant solids by filtration, rinsing the solids with an anti-solvent, and then drying the solids to prepare a 1:1 diastereomeric mixture of the Formula (1) compound.

8. The process of claim 7 comprising the steps of:
a) pre-activating the Formula (B) compound with a palladium catalyst selected from the group consisting of Pd(OAc)₂, PdCl₂, Pd-G2-XPhos, Pd-XPhos Crotyl Cl, Pd(dppf)Cl₂, Pd-G2-PCy₃, and Pd₂(dba)₃, in the presence of an alcohol, a ligand selected from the group consisting of XPhos, SPhos, dppp, dppf, dba, PPh₃, and PCy₃, and a borylation buffer that comprises an acid and a base, wherein said acid is selected from the group consisting of HOAc, citric acid, formic acid, chloroacetic acid, and ammonium acetate;
b) borylating the preactivated Formula (B) compound of Step (a) by adding a borylating agent selected from the group consisting of bisboronic acid, bisboronic acid and ethylene glycol; bisboronic acid and propylene glycol; B₂Pin₂, and B₂(NMe₂)₄ in an alcohol;
c) mixing the Formula (A) compound with a base in an alcohol, co-solvent, or mixtures thereof; and wherein the base in Step (a) and (c) is selected from the group consisting of KOAc, CsOAc, TEA, K₂CO₃, Na₂CO₃, Cs₂CO₃, DIPEA, and K₃PO₄; and wherein the alcohol in Steps (a) and (B) in anhydrous ethanol;
d) mixing the reactants of Step (c) to the reactants of Step (b) to give the compound of Formula (1), and diastereomers thereof;
e) purifying the Formula (1) compound of Step (d) by concentrating the reaction product of Step (d) and extracting the Formula (1) compound into an extracting solvent selected from the group consisting of THF, EtOAc, MeOAc, methylene chloride and MeTHF;
f) adding an aqueous wash to the extraction of step (e) selected from water or brine, each of which contain a palladium chelator selected from the group consisting of EDA, TMT-Na₃, NH₄OH, TMT, NaHSO₃, thiourea, DEA, EDTA, Ac-L-cysteine, citric acid and mixtures thereof, and separating the organic layer.
g) adding a palladium scavenger to the organic layer of step (f), or recycling the organic layer of step (f) through a cartridge of metal scavenger selected from the group consisting of Carbon with EDA, Silica gel with EDA, Si-Thiol, MP-TU, MP-TMT, Si-TMT, Si-DMT, and Si-cysteine, filtering out the solids, rinsing the solids with an extraction solvent(s) and concentrating the filtrate;
h) dissolving the resulting concentrate of Step (g) in an organic solvent selected from the group consisting of MEK, iPrOAc, EtOAc, acetone, 1-butanol, 1-propanol, 2-propanol, and mixtures thereof with heat; cooling and seeding the solution with the Formula (1) compound; and
i) cooling the mixture of step (h), optionally adding an anti-solvent selected from the group consisting of water, MTBE, hexane, heptane, and mixtures thereof; collecting the resultant solids by filtration, rinsing the solids with the anti-solvent, and then drying the solids to prepare a 1:1 diastereomeric mixture of the Formula (1) compound.

9. The process of claim 8, wherein the co-solvent is selected from the group consisting of iPrOAc, EtOAc, DMF, DME, THF, MeTHF, and acetonitrile, and mixtures thereof; and wherein the palladium catalyst in Step (a) is Pd(OAc)₂; and wherein the ligand in Step (a) is XPhos and the borylation buffer in Step (a) is an alcohol solution comprising HOAc and a base; and wherein the borylating agent in Step (b) is bisboronic acid and ethylene glycol in anhydrous ethanol.

10. The process of claim 9, wherein the base in Step (a) is KOAc and the base in Step (c) is selected from the group consisting of K₂CO₃, Na₂CO₃, TEA, and mixtures thereof.

11. The process of claim 10, wherein the extracting solvent in Step (e) is THF; and wherein the organics from Step (e) are washed 1×, 2×, 3×, or 4× with the aqueous wash; and wherein the palladium scavenger in Step (g) is Carbon with EDA and the organic solvent is selected from the group consisting of MEK, IPA, EtOAc, acetone, 1-butanol, 1-propanol, 2-propanol, and mixtures thereof; and wherein the anti-solvent in Step (h) is selected from the group selected from water, MTBE, hexane, heptane, and mixtures thereof; and the solutions added to the reaction in Steps (a-c) are purged with N₂ or Ar and the reaction in Steps (a-d) occurs under a N₂ or Ar atmosphere.

12. The process of claim 11 wherein the organic solvent is MEK, 1-propanol, or a mixture thereof; and the anti-solvent is heptane.

13. A process for preparing the Formula (1) compound, 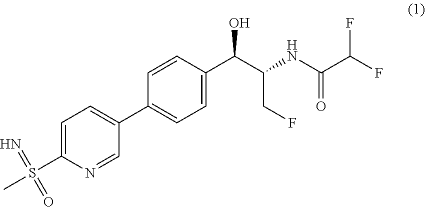 [SMILES: CS(=N)(=O)c1ccc(-c2ccc([C@@H](O)[C@@H](CF)NC(=O)C(F)F)cc2)cn1]
comprising the steps of:
a) pre-activating the Formula (B) compound with a palladium catalyst selected from the group consisting of Pd(OAc)₂, PdCl₂, Pd-G2-XPhos, Pd-XPhos Crotyl Cl, Pd(dppf)Cl₂, Pd-G2-PCy₃, and Pd₂(dba)₃; in the presence of an alcohol, a ligand selected from the group consisting of XPhos, SPhos, dppp, dppf, dba, PPh₃, and PCy₃; and a borylation buffer; 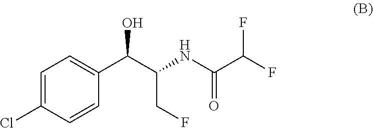 [SMILES: O=C(N[C@H](CF)[C@H](O)c1ccc(Cl)cc1)C(F)F]
b) borylating the preactivated Formula (B) compound of Step (a) by adding a borylating agent selected from the group consisting of bisboronic acid, bisboronic acid and ethylene glycol; bisboronic acid and propylene glycol; B₂Pin₂, and B₂(NMe₂)₄ in an alcohol;
c) mixing the Formula (A) compound with a base in an alcohol, co-solvent, or mixtures thereof; 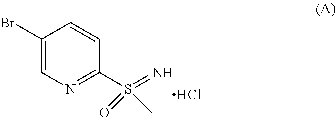
d) adding the reactants of Step (c) to the reactants of Step (b) to give the Formula (1) compound, and diastereomers thereof.

14. The process of claim 13, wherein the borylation buffer in step (a) comprises an acid and a base, wherein the acid is selected from the group consisting of HOAc, citric acid, formic acid, chloroacetic acid, and ammonium acetate; and the base is selected from the group consisting of KOAc, CsOAc, TEA, K₂CO₃, Na₂CO₃, Cs₂CO₃, DIPEA, and K₃PO₄.

15. The process of claim 14, wherein the alcohol is anhydrous ethanol and the base in step (a) is KOAc and the base in step (c) is selected from the group consisting of K₂CO₃, Na₂CO₃, TEA, and mixtures thereof; and wherein the resultant Formula (1) compound is concentrated.

16. The process of claim 15, wherein the Formula (1) compound is purified by extracting the compound into an extracting solvent selected from the group consisting of THF, EtOAc, MeOAc, methylene chloride and MeTHF.

17. The process of claim 16, wherein the purified Formula (1) compound is washed with an aqueous wash of water or brine each of which contain a palladium chelator selected from the group consisting of EDA, TMT-Na₃, NH₄OH, TMT, NaHSO₃, thiourea, DEA, EDTA, Ac-L-cysteine, citric acid and mixtures thereof, and separating the organic layer.

18. The process of claim 17, further comprising the addition of a palladium scavenger that is Carbon with EDA to the organic layer or recycling the organic layer through a metal scavenger cartridge consisting of Carbon with EDA, Silica gel with EDA, Si-Thiol, MP-TU, MP-TMT, Si-TMT, Si-DMT, and Si-cysteine; filtering out the solids, rinsing the solids with an extraction solvent(s) and concentrating the filtrate.

19. The process of claim 18, wherein the concentrated filtrate is dissolved in an organic solvent selected from the group consisting of MEK, iPrOAc, EtOAc, acetone, 1-butanol, 1-propanol, 2-propanol, and mixtures thereof with heat; cooling and seeding the solution with the Formula (1) compound.

20. The process of claim 19, wherein the resulting mixture is cooled and optionally further comprising the addition of an anti-solvent selected from the group conbsisting of water, MTBE, hexane, heptane, and mixtures thereof; collecting the resultant solids by filtration, rinsing the solids with the anti-solvent, and then drying the solids to prepare a 1:1 diastereomeric mixture of the Formula (1) compound.
